(19) United States
(12) Patent Application Publication
(10) Pub. No.: US 2016/0220270 A1
(43) Pub. Date: Aug. 4, 2016

(54) PUNCTURE DEVICE

(71) Applicants: Mitsubishi Pencil Company, Limited, Tokyo (JP); Terumo Kabushiki Kaisha, Tokyo (JP)

(72) Inventors: Makoto TAMURA, Fujioka-shi (JP); Hisami TAMANO, Fujioka-shi (JP); Mitsuhiro KAWABATA, Fujioka-shi (JP)

(73) Assignees: Mitsubishi Pencil Company, Limited, Tokyo (JP); Terumo Kabushiki Kaisha, Tokyo (JP)

(21) Appl. No.: 15/012,194

(22) Filed: Feb. 1, 2016

Related U.S. Application Data
(63) Continuation of application No. PCT/JP2014/070381, filed on Aug. 1, 2014.

(30) Foreign Application Priority Data
Aug. 5, 2013 (JP) 2013-162333
Jan. 23, 2014 (JP) 2014-010315

(51) Int. Cl. A61B 17/34

(52) U.S. Cl. CPC A61B 17/3421; A61B 17/3415; A61B 2017/347

(57) ABSTRACT

To provide a puncture device in which an inner needle (3) is accommodated in an inner needle hub (41) by pulling operation of an inner needle, a through hole for inserting the inner needle is blocked, and the inner needle is prevented from protruding outside. The puncture device includes an outer pipe (6) fitted inside the inner needle hub (41) so as to be freely moveable, and an inner pipe (7) fitted inside the outer pipe (6) so as to be freely moveable, which has a through hole (73) for inserting the inner needle (3). The inner pipe (7) includes a through hole block means (space 75, the block body 74) for blocking communicated condition of the through hole, which makes at least part of the through hole move from a position where the inner needle (3) is inserted to a position where the inner needle (3) is blocked.

### TECHNICAL FIELD

The present invention relates to a puncture device, and more particularly to a puncture device for indwelling an outer needle (catheter) in a blood vessel.

### BACKGROUND ART

Conventionally, when a puncture device for indwelling an outer needle (catheter) in a blood vessel is used, there is a possibility of stabbing the used inner needle which is pulled from a blood vessel into the user himself/herself, a patient's body, or an industrial waste disposal worker and causing hepatitis or communicable disease. Therefore a safety measure for the used inner needle has been desired.

As a safety measure, for example, Patent Document 1 discloses a puncture device which includes a needle body providing a sharp needle tip at the end, a hub fixed to a base of the needle body, a protector disposed relatively movable to the needle body and capable of storing the needle tip of the needle body, a shutter operation means which includes a contacting member and a coil spring (a movement means), and a rope (a removal prevent means). The above mentioned puncture device has a structure that when a compressed coil spring is released, a protector body slides and protects the needle tip fixed to the hub.

However, in the puncture device disclosed in the Patent Document 1, a user (operator) has to work the compressed coil spring to protect the needle tip. If the user forget to do so, a needle contaminated with blood lead to be exposed. Therefore, there is a problem that safety is not secured.

A puncture for solving the above mentioned problem is disclosed in Patent document 2 and Patent Document 3. The puncture disclosed in the Patent Documents 2 and 3 includes an outer needle, an outer needle hub for retaining the outer needle, an inner needle whose tip portion is inserted in the above-mentioned outer needle, and a cylindrical inner needle hub for retaining a base portion of the above-mentioned inner needle at its inside, and the puncture has a structure that after the outer needle is indwelled inside a blood vessel, the inner needle is accommodated in the inner needle hub and the accommodated inner needle is prevented from protruding out of the inner needle hub.

Specifically, the puncture further includes a needle block member for blocking an internal passage for the inner needle, which consists of an elastic member. When the inner needle is accommodated in the inner needle hub, restoring force of the needle block member blocks the internal passage for the inner needle thereby the accommodated inner needle is prevented from protruding out of the inner needle hub.

As described above, as for the puncture disclosed in the Patent Documents 2 and 3, after the outer needle is indwelled inside a blood vessel, a user (operator) pulls the inner needle out of the outer needle, which accompanies that the inner needle is accommodated in the inner needle hub and the accommodated inner needle is prevented from protruding out of the inner needle hub. Therefore it is possible to prevent a situation that a needle is exposed when the user (operator) forgets to do an operation for protecting the needle tip as disclosed in the Patent Document 1 and it is possible to secure safety.

### PRIOR ART DOCUMENTS

### Patent Documents

Patent Document 1: Japanese patent laid-open No. 2002-28236
Patent Document 2: Japanese patent laid-open No. H11-57002
Patent Document 3: Japanese patent laid-open No. 2002-126080

### Problems to be Solved by the Invention

Incidentally, in the puncture device disclosed in Patent Documents 2 and 3 above, the needle block member is in pressure contact with an outer surface of the inner needle by restoring force (elastic force) of the needle block member in a situation where the inner needle is in the internal passage for the inner needle. When the inner needle passes through a position of the needle block member on a pulling operation, the needle block member expands in the internal passage by restoring force (elastic force) of the needle block member and the internal passage is blocked.

There is a problem that when the user (operator) pulls the inner needle from the outer needle, the restoring force (elastic force) of the needle block member becomes sliding resistance since the needle block member is in pressure contact with an outer surface of the inner needle, and the pulling is not easy for the user.

If the restoring force (elastic force) of the needle block member is set to be small for reducing the sliding resistance at the pulling operation, the needle block member may fail to expand in the internal passage enough and not completely block the internal passage. Therefore the inner needle may protrude outside.

The needle block member is formed of a synthetic resin material such as polyethylene or a metal material such as stainless steel. The needle block member formed of stainless steel is not good because it is too costly.

On the other hand, the needle block member formed of a synthetic resin material is preferable in cost but there is a possibility that the internal passage may not be blocked enough when the inner needle is pulled and the inner needle may protrude outside. In a situation that the needle block member is in pressure contact with an outer surface of the inner needle (before the inner needle is pulled out of the outer needle) the needle block member is in a deformed state. Therefore in case the puncture device is not used for a long time because of long storage, plastic deformation may occurs in the needle block member, the needle block member may not expand enough in an inner needle pulling operation and may fail to block the internal passage, and the inner needle may protrude outside.

In order to solve the above-mentioned problems, the present inventors have made an effort to provide a present invention that is, a puncture device in which sliding resistance at the inner needle pulling operation is reduced as much as possible, which is produced at a low cost, and in which an inner needle is prevented from protruding outside after the inner needle pulled from an outer needle is accommodated in an inner needle hub even if the puncture device is not used for a long time.

The present invention has been made in order to solve the above-mentioned technical problems, and aims to provide a puncture device in which an inner needle is accommodated in an inner needle hub by a pulling operation, a through hole for insertion of the inner needle is blocked without using restoring force of a needle block member and the inner needle is prevented from protruding outside.

### Means for Solving the Problems

The puncture device in accordance with the present invention made in order to solve the above-mentioned technical problems is a puncture device provided with an outer needle, an outer needle hub for retaining a base portion of the above-mentioned outer needle, an inner needle whose tip portion is inserted in the above-mentioned outer needle, and a cylindrical syringe for retaining the base portion of the above-mentioned inner needle; the above-mentioned syringe is provided with an inner needle hub for retaining a base portion of the above-mentioned inner needle and a cylinder body which has gripping means for gripping the above-mentioned outer needle hub and is mounted inside the above-mentioned inner needle hub to move back and forth; the above-mentioned cylinder body is provided with an outer pipe mounted inside the above-mentioned inner needle hub so as to be moveable to and fro, and an inner pipe which is mounted inside the above-mentioned outer pipe so as to be moveable to and fro and is provided with a through hole into which the above-mentioned inner needle is inserted; either the inner pipe or the inner pipe and the outer pipe is provided with through hole block means for blocking communicated condition of the through hole, which makes at least part of the through hole move from a position where the inner needle is inserted to a position where the inner needle is blocked wherein insertion of the inner needle is blocked by moving the through hole of the inner pipe with the through hole block means.

As described above, the present invention in which a conventional needle block member is not used but is characterized in that at least part of the through hole of the inner pipe is moved from a position where the inner needle is inserted to a position where the inner needle is blocked whereby a communication state of the through hole is blocked and insertion of the inner needle is blocked. Specifically, the preset invention does not use restoring force for blocking the through hole as opposed to a conventional needle block member. Therefore, in case the puncture device is not used for a long time for reasons of long storage, plastic deformation does not occur and it is possible to block the communication state of the through hole of the inner pipe reliably. In addition, as a needle block member of the present invention is not needed in pressure contact with the inner needle unlike in the case of the conventional needle block member, it is possible to reduce sliding resistance at a pulling operation thereby user pulls the inner needle easily.

Here, it is preferable that the through hole block means includes a block body in which a through hole for inserting the inner needle is formed, and a space part which movably accommodates the block body and has a structure that when a tip of the inner needle is pulled out through the block body, the block body moves in the space part by the block body's own weight and insertion of the inner needle is blocked.

When the tip of the inner needle is pulled out through the block body, the block body moves in the space part by the block body's own weight whereby the through hole of the inner pipe is blocked. Therefore it is possible to prevent the inner needle accommodated in the inner needle hub or, inner needle hub and the cylinder body from protruding outside.

In addition, since the restoring force is not used in this invention in contrast to the conventional needle block member, even if the puncture device is not used for a long time for reasons of long storage, plastic deformation does not occur and it is possible to block the communication state of the through hole of the inner pipe reliably.

Further, since a size of a through hole of the block body is formed slightly larger than an inner needle diameter, it is possible to reduce the sliding resistance at the pulling operation of the inner needle and the pulling is easy for the user.

Further, it is preferable that the inner pipe is provided with a standing piece which rises with being forced out by the inner needle in the through hole and engages with the outer pipe, and when the inner needle pulled out passing through the standing piece, the standing piece is disengaged with the outer pipe, and the disengaged standing piece blocks insertion of the inner needle.

According to above, communicated condition of the through hole is blocked more reliably by the disengaged standing piece whereby the inner needle is surely prevented from protruding outside.

Here, it is preferable that at least the inner pipe and the block body are made of synthetic resin.

It is possible to produce the inner pipe and the block at a low cost by using synthetic resin.

It is preferable that the through hole block means includes a groove having at least a spiral portion, which is formed on an inner surface of the outer pipe or an outer surface of the inner pipe and a projecting part movable in the groove, which is formed on an outer surface of the inner pipe or an inner surface of the outer pipe and the through hole in the inner pipe is formed at away from the central axis of the inner pipe, and when the outer pipe moves relative to the inner pipe, the inner pipe and the outer pipe relatively rotate by engagement of the groove and the projecting part, whereby the through hole of the inner pipe moves from a position where the inner needle is inserted to a position where the inner needle is blocked whereby insertion of the inner needle is blocked.

As described above, when the outer pipe moves relative to the inner pipe, the inner pipe and the outer pipe relatively rotate, the through hole of the inner needle moves from a position where the inner needle is inserted to a position where the inner needle is blocked and insertion of the inner needle is blocked. Consequently, this motion of the cylinder body can prevent the inner needle accommodated inside the above-mentioned inner needle hub or inside the inner needle hub from protruding outside.

In addition, since the restoring force is not used in this invention in contrast to the conventional needle block member, even if the puncture device is not used for a long time for reasons of long storage, plastic deformation does not occur and it is possible to prevent the inner needle from protruding outside.

Further, since a size of a through hole of the inner pipe is formed slightly larger than an inner needle diameter, it is possible to reduce the sliding resistance at the pulling operation of the inner needle and the pulling is easy for the user.

Further, it is preferable that the inner pipe is provided with a standing piece which rises with being forced out by the inner needle in the through hole and engages with the outer pipe, and when the inner needle pulled out passing through the standing piece, the standing piece is disengaged with the outer pipe, and the disengaged standing piece blocks insertion of the inner needle.

Furthermore, it is preferable that the outer pipe and the inner pipe are made of synthetic resin.

It is preferable that the through hole block means includes a projection formed on an inner surface of the outer pipe, a block body having a through hole for insertion of the inner needle, a space part formed in the inner pipe, in which the block body movably accommodated, and when the outer pipe moves relative to the inner pipe, the block body is moved in the space part by the projection formed on an inner surface of the outer pipe whereby the insertion of the inner needle is blocked.

According to the above mentioned through hole block means, the block body is moved in the space part by the projection formed on an inner surface of the outer pipe in accordance with the outer pipe moving relative to the inner pipe and the insertion of the inner needle is blocked. Therefore, it is possible to prevent the inner needle from protruding outside.

In addition, since restoring force is not used as opposed to a conventional needle block member, incase the puncture device is not used for a longtime for reasons of long storage, plastic deformation does not occur and it is possible to block the communication state of the through hole of the inner pipe reliably.

Further, since a size of a through hole of the block body is formed slightly larger than an inner needle diameter, it is possible to reduce the sliding resistance at the pulling operation of the inner needle and the pulling is easy for the user.

It is preferable that a part to be engaged formed on the block body is engaged to an engaging part formed in the inner pipe so that movement of the block body is prevented and a state that the insertion of the inner needle is blocked is kept.

As described above, a block body movement prevention means is formed so that the state that the insertion of the inner needle is blocked is kept and it is possible to block the insertion of the inner needle more reliably.

In addition, it is preferable that the outer pipe, the inner pipe, and the block body are made of synthetic resin. If the outer pipe, the inner pipe, and the block body are made of synthetic resin such as polyethylene, it is possible to produce them at a low cost.

### Effects of the Invention

According to the present invention, a puncture device can be obtained in which an inner needle is accommodated in an inner needle hub and the like by pulling operation of an inner needle, and a through hole for inserting the inner needle is blocked without using restoring force, and which prevents the inner needle from protruding outside.

### BRIEF DESCRIPTION OF THE DRAWINGS

FIG. 1 is a perspective view showing an appearance of a puncture device in accordance with a first preferred embodiment of the present invention.

FIG. 2 is a perspective view showing a situation where a protector of the puncture device in FIG. 1 is removed.

FIG. 3 is an exploded perspective view except the protector of the puncture device in FIG. 1.

FIG. 4 is a longitudinal sectional view showing the puncture device shown in FIG. 1.

FIG. 5 is a longitudinal sectional view showing the puncture device of FIG. 1, and is a longitudinal sectional view whose section is different from that of the longitudinal sectional view of FIG. 4 by 90 degrees in a circumferential direction.

FIG. 6A is a perspective view showing an outer pipe.

FIG. 6B is a longitudinal sectional view showing the outer pipe.

FIG. 6C is a longitudinal sectional view showing the outer pipe whose section is different from that of the longitudinal sectional view of FIG. 6B by 90 degrees in a circumferential direction.

FIG. 7 is the perspective view showing an inner pipe.

FIG. 8A is a plan view showing the inner pipe in an unfolded configuration.

FIG. 8B is a sectional view along the line A-A of FIG. 8A showing the inner pipe in the unfolded configuration.

FIG. 8C is a rear view of FIG. 8A showing the inner pipe in the unfolded configuration.

FIG. 9 is a perspective view showing a block body.

FIG. 10A is a longitudinal sectional view showing a situation where the expansion of the inner needle hub is completed, for explaining the procedure for use of the puncture device shown in FIG. 1.

FIG. 10B is a longitudinal sectional view whose section is different from that of the longitudinal sectional view of FIG. 10A by 90 degrees in a circumferential direction, showing the situation where the expansion of the inner needle hub is completed, for explaining the procedure for use of the puncture device shown in FIG. 1.

FIG. 11A is a longitudinal sectional view showing a situation where the outer needle is released (unlocked), for explaining the procedure for use of the puncture device shown in FIG. 1.

FIG. 11B is a longitudinal sectional view whose section is different from that of the longitudinal sectional view of FIG. 11A by 90 degrees in a circumferential direction, showing the situation where the outer needle is released (unlocked), for explaining the procedure for use of the puncture device shown in FIG. 1.

FIG. 12 is a perspective view showing an appearance of a puncture device in accordance with a second preferred embodiment in a situation where a protector is removed.

FIG. 13A is a longitudinal sectional view showing a puncture device in accordance with the second preferred embodiment of the present invention.

FIG. 13B is a longitudinal sectional view whose section is different from that of the longitudinal sectional view of FIG. 13A by 90 degrees in a circumferential direction, showing the puncture device in accordance with the second preferred embodiment of the present invention.

FIG. 14A is a perspective view showing the outer pipe in FIGS. 13A and 13B.

FIG. 14B is a longitudinal sectional view showing the outer pipe in FIGS. 13A and 13B.

FIG. 15A is a perspective view showing the inner pipe in FIGS. 13A and 13B.

FIG. 15B is a plan view showing the inner pipe in FIGS. 13A and 13B.

FIG. 15C is a longitudinal sectional view showing the inner pipe in FIGS. 13A and 13B.

FIG. 16A is a longitudinal sectional view showing a situation where the expansion of the inner needle hub is completed, for explaining the procedure which uses the puncture device in accordance with the second preferred embodiment of the present invention.

FIG. 16B is a longitudinal sectional view whose section is different from that of the longitudinal sectional view of FIG. 16A by 90 degrees in a circumferential direction, showing the situation where the expansion of the inner needle hub is completed, for explaining the procedure which uses the puncture device in accordance with the second preferred embodiment of the present invention.

FIG. 17A is a longitudinal sectional view showing a situation where the expansion of a relay pipe is completed, for explaining the procedure for use of the puncture device in accordance with the second preferred embodiment of the present invention.

FIG. 17B is a longitudinal sectional view whose section is different from that of the longitudinal sectional view of FIG. 17A by 90 degrees in a circumferential direction, showing the situation where the expansion of a relay pipe is completed, for explaining the procedure for use of the puncture device in accordance with the second preferred embodiment of the present invention.

FIG. 18A is a longitudinal sectional view showing a situation where the inner pipe is passing through a straight portion of the outer pipe, for explaining the procedure which uses the puncture device in accordance with the second preferred embodiment of the present invention.

FIG. 18B is a longitudinal sectional view whose section is different from that of the longitudinal sectional view of FIG. 18A by 90 degrees in a circumferential direction, showing the situation where the inner pipe is passing through a straight portion of the outer pipe, for explaining the procedure which uses the puncture device in accordance with the second preferred embodiment of the present invention.

FIG. 19A is a longitudinal sectional view showing a situation where the inner pipe is passing through a spiral portion of the outer pipe, for explaining the procedure for use of the puncture device in accordance with the second preferred embodiment of the present invention.

FIG. 19B is a longitudinal sectional view whose section is different from that of the longitudinal sectional view of FIG. 19A by 90 degrees in a circumferential direction, showing the situation where the inner pipe is passing through the spiral portion of the outer pipe, for explaining the procedure for use of the puncture device in accordance with the second preferred embodiment of the present invention.

FIG. 20 is a sectional view showing a situation where the outer needle is released (unlocked), for explaining the procedure for use of the puncture device in accordance with the second preferred embodiment of the present invention.

FIG. 21 is an exploded perspective view of the puncture device (except the protector) in accordance with a third preferred embodiment of the present invention.

FIG. 22A is a perspective view showing the outer pipe in FIG. 21.

FIG. 22B is a longitudinal sectional view showing the outer pipe in FIG. 21.

FIG. 22C is a longitudinal sectional view showing the outer pipe in FIG. 21 whose section is different from that of the longitudinal sectional view of FIG. 22B by 90 degrees in a circumferential direction.

FIG. 23A is a perspective view showing the inner pipe in FIG. 21.

FIG. 23B is a perspective view from the bottom side of FIG. 23A showing the inner pipe in FIG. 21.

FIG. 24A is a plan view showing the inner pipe in FIG. 21.

FIG. 24B is a bottom view showing the inner pipe in FIG. 21.

FIG. 25A is a sectional view along the line A-A of FIG. 24A.

FIG. 25B is a sectional view along the line B-B of FIG. 24A.

FIG. 26A is a perspective view showing the block body in FIG. 21.

FIG. 26B is a side view showing the block body in FIG. 21.

FIG. 26C is a front view showing the block body in FIG. 21.

FIG. 27A is a longitudinal sectional view showing the puncture device in FIG. 21.

FIG. 27B is a longitudinal sectional view showing the puncture device in FIG. 21 whose section is different from that of the longitudinal sectional view of FIG. 27A by 90 degrees in a circumferential direction.

FIG. 28A is a longitudinal sectional view showing a situation where the expansion of the inner needle hub is completed, for explaining the procedure for use of the puncture device shown in FIG. 21.

FIG. 28B is a longitudinal sectional view whose section is different from that of the longitudinal sectional view of FIG. 28A by 90 degrees in a circumferential direction, showing the situation where the expansion of the inner needle hub is completed, for explaining the procedure for use of the puncture device shown in FIG. 21.

FIG. 29A is a longitudinal sectional view showing a situation where the outer needle is released (unlocked), for explaining the procedure for use of the puncture device shown in FIG. 21.

FIG. 29B is a longitudinal sectional view whose section is different from that of the longitudinal sectional view of FIG. 29A by 90 degrees in a circumferential direction, showing the situation where the outer needle is released (unlocked), for explaining the procedure for use of the puncture device shown in FIG. 21.

FIG. 29C is a sectional view along the line A-A of FIG. 29A showing the situation where the outer needle is released (unlocked), for explaining the procedure for use of the puncture device shown in FIG. 21.

FIG. 30A is a perspective view showing an inner pipe of the puncture device in accordance with a fourth preferred embodiment of the present invention.

FIG. 30B is a plan view showing the inner pipe of the puncture device in accordance with a fourth preferred embodiment of the present invention.

FIG. 30C is a longitudinal sectional view showing the inner pipe of the puncture device in accordance with a fourth preferred embodiment of the present invention.

FIG. 31A is a plan view showing the inner pipe in an unfolded configuration.

FIG. 31B is a sectional view along the line A-A of FIG. 31A showing the inner pipe in the unfolded configuration.

FIG. 31C is a bottom view of FIG. 31A showing the inner pipe in the unfolded configuration.

FIG. 32 is a perspective view of a block body of the puncture device in accordance with the fourth preferred embodiment of the present invention.

FIG. 33 is a plane view of the block body shown in FIG. 32.

FIG. 34 is a longitudinal sectional view of the block body shown in FIG. 32.

FIG. 35A is a longitudinal sectional view showing the puncture device in accordance with a fourth preferred embodiment of the present invention.

FIG. 35B is a longitudinal sectional view whose section is different from that of the longitudinal sectional view of FIG. 35A by 90 degrees in a circumferential direction, showing the puncture device in accordance with a fourth preferred embodiment of the present invention.

FIG. 36A is a longitudinal sectional view showing a situation where the expansion of the inner needle hub is completed, for explaining the procedure which uses the puncture device shown in FIGS. 35A and 35B.

FIG. 36B is a longitudinal sectional view whose section is different from that of the longitudinal sectional view of FIG. 36A by 90 degrees in a circumferential direction, showing the situation where the expansion of the inner needle hub is completed, for explaining the procedure which uses the puncture device shown in FIGS. 35A and 35B.

FIG. 37A is a longitudinal sectional view showing a situation where the outer needle is released (unlocked), for explaining the procedure which uses the puncture device shown in FIGS. 35A and 35B.

FIG. 37B is a longitudinal sectional view whose section is different from that of the longitudinal sectional view of FIG. 37A by 90 degrees in a circumferential direction, showing a situation where the outer needle is released (unlocked), for explaining the procedure which uses the puncture device shown in FIGS. 35A and 35B.

### MODE FOR CARRYING OUT THE INVENTION

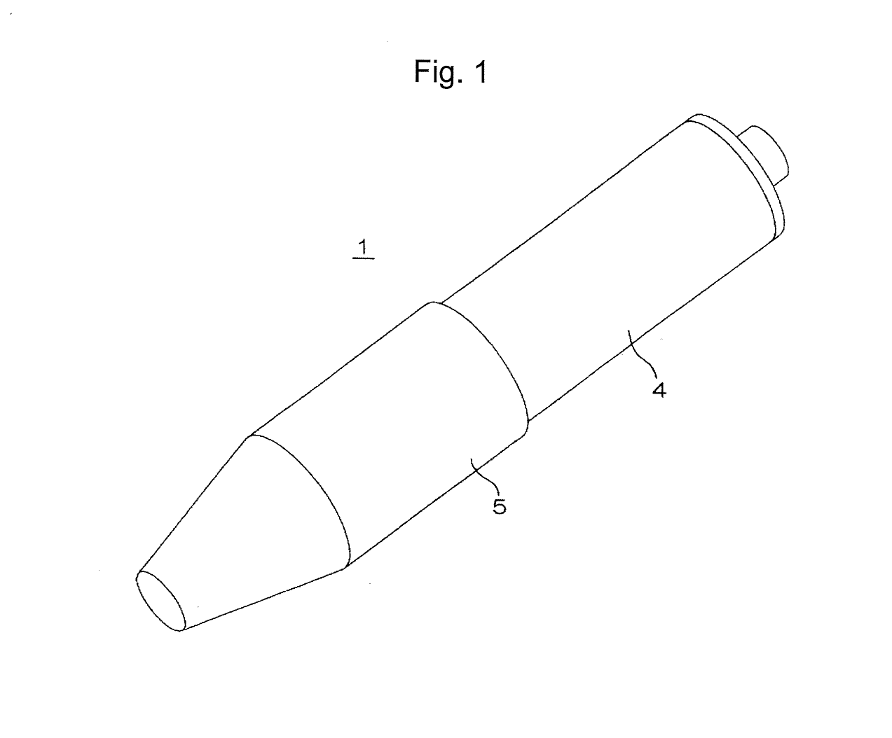

Hereinafter, a puncture device in accordance with a first preferred embodiment of the present invention will be described with reference to FIGS. 1 to 11.

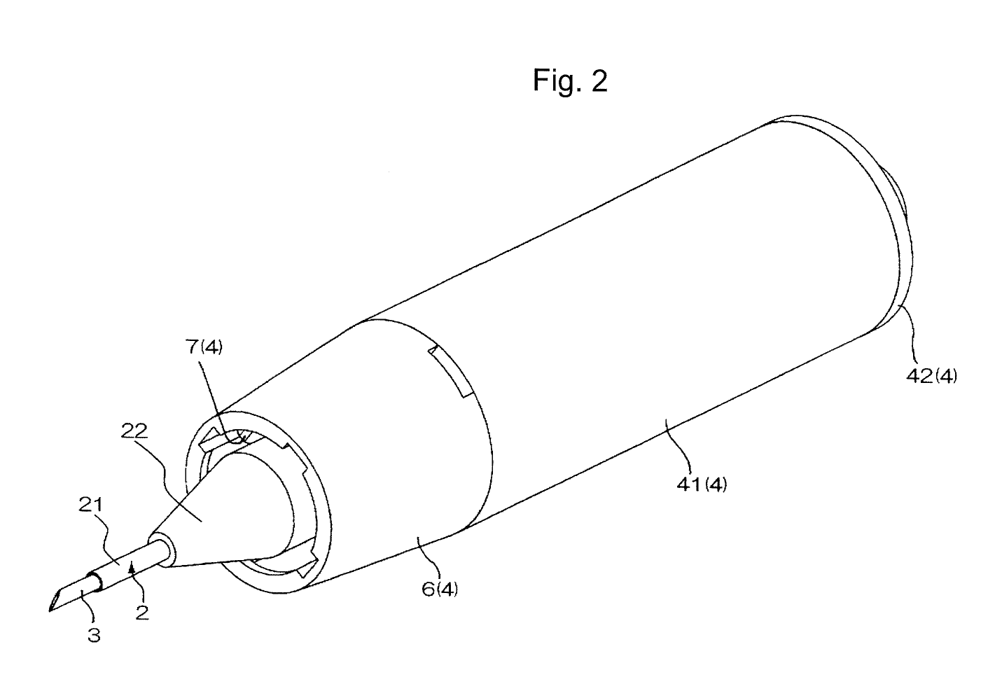

As shown in FIGS. 1 and 2, a puncture device 1 is provided with an outer needle 21, an inner needle 3 whose tip portion (left-hand side) is inserted in the above-mentioned outer needle 21, a cylindrical syringe 4 for retaining an end portion (base portion) of the above-mentioned inner needle 3, and a protector 5 which covers the above-mentioned outer needle 21 and the above-mentioned inner needle 3. Further, in the above-mentioned puncture device 1, all the components except the above-mentioned inner needle 3 are made of resin. It should be noted that the above-mentioned outer needle 21 and the above-mentioned inner needle 3 cannot be seen in FIG. 1, since they are covered with the above-mentioned protector 5.

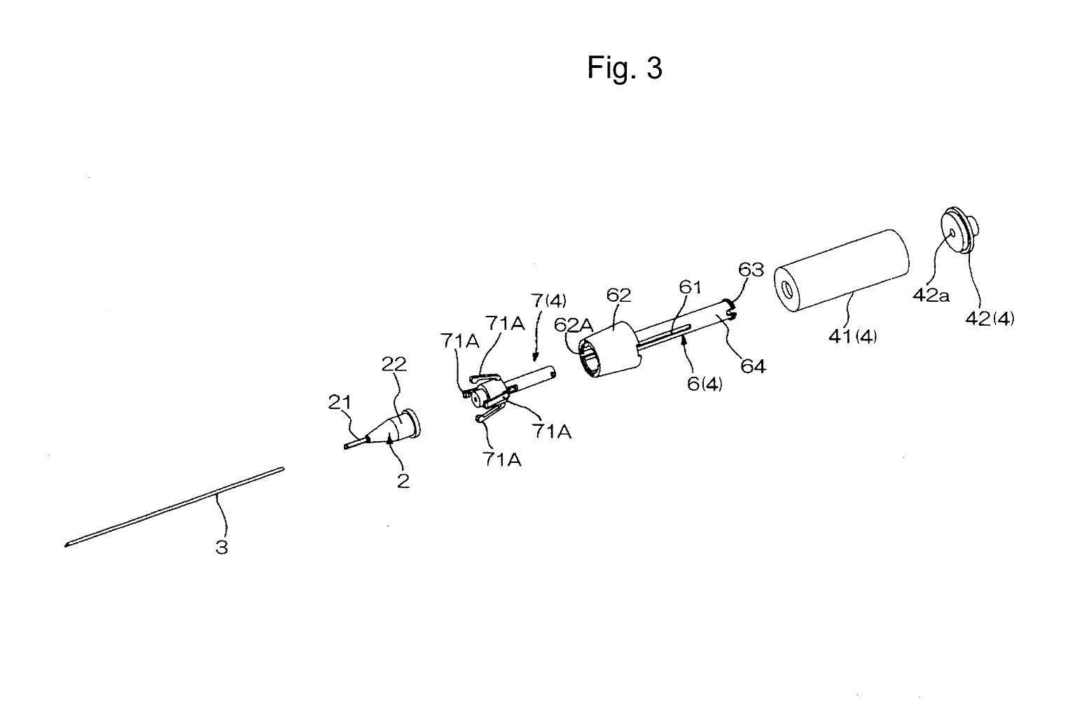

Further, as shown in FIGS. 2 and 3, the above-mentioned catheter 2 has the outer needle 21 formed of a flexible hollow pipe and the outer needle hub 22 for retaining the base portion of the outer needle 21.

As shown in FIGS. 2 to 5, the above-mentioned syringe 4 is provided with a cylindrical inner needle hub 41 and a plug 42 which is press fitted into and attached to a base portion (right-hand side) of the above-mentioned inner needle hub 41 and has a substantially cylindrical needle retaining part 42a by which the base portion of the above-mentioned inner needle 3 is retained.

Further, the above-mentioned syringe 4 is provided with an outer pipe 6 fitted inside the above-mentioned inner needle hub 41 so as to be freely moveable, and an inner pipe 7 having four arms 71A for gripping the above-mentioned outer needle hub 22 and fitted inside the above-mentioned outer pipe 6 so as to be freely moveable.

It should be noted that although this preferred embodiment shows a situation where the above-mentioned four arms 71A are formed at the above-mentioned inner pipe 7, the outer needle hub 22 may be retained by two or more arms.

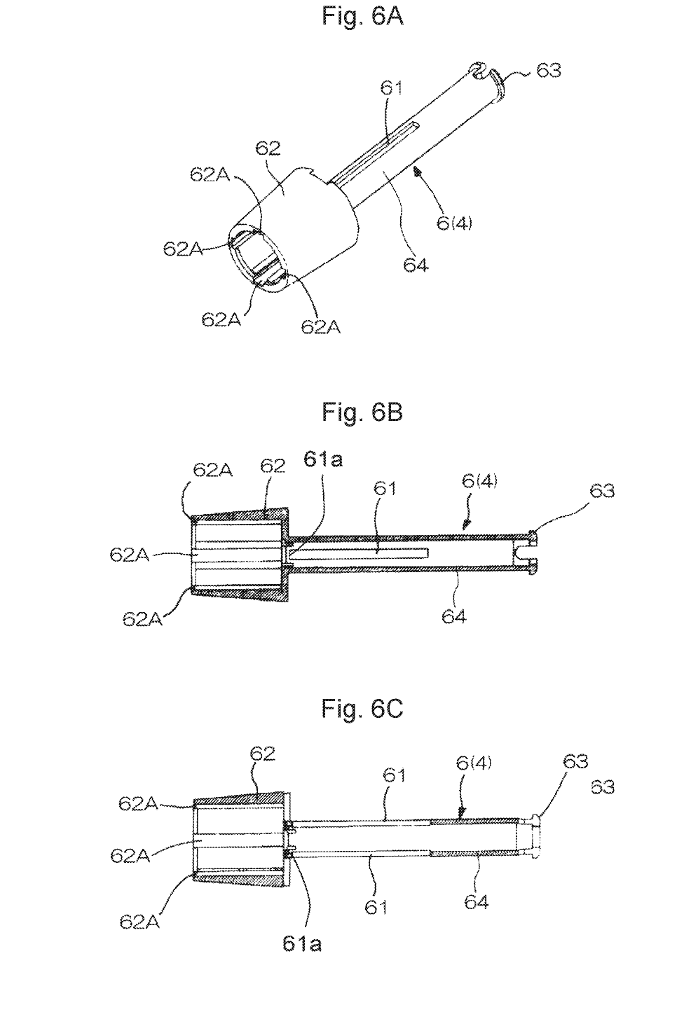

As shown in FIGS. 6A, 6B, and 6C, the above-mentioned outer pipe 6 is provided with a groove 61 formed along an axis of a shaft 64 of the above-mentioned outer pipe 6 (formed along a direction of back and forth movement of the inner pipe 7) and an arm opening/closing part 62 which accommodates the above-mentioned four arms 71A, and cylindrically formed as a whole.

It should be noted that the above-mentioned four arms 71A are arranged to be moveable guided by four guide grooves 62A formed at upper and lower sides, and both lateral sides on the inner surface of the above-mentioned arm opening/closing part 62.

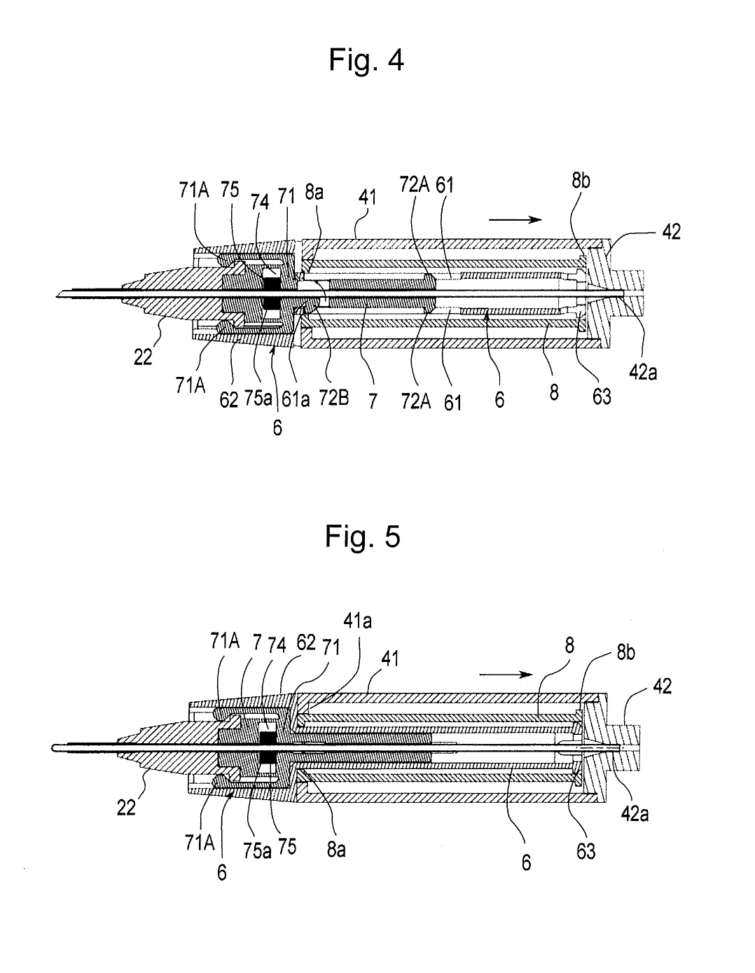

As shown in FIG. 4 and FIG. 6C, the above-mentioned grooves 61 are axisymmetrically formed at two places, an upper part and a lower part in the periphery of the above-mentioned outer pipe 6.

Further, as shown in FIGS. 4 and 5, when the above-mentioned inner pipe 7 is accommodated in the above-mentioned outer pipe 6, the above-mentioned four arms 71A are folded, and the above-mentioned arm opening/closing part 62 grasps the above-mentioned outer needle hub 22.

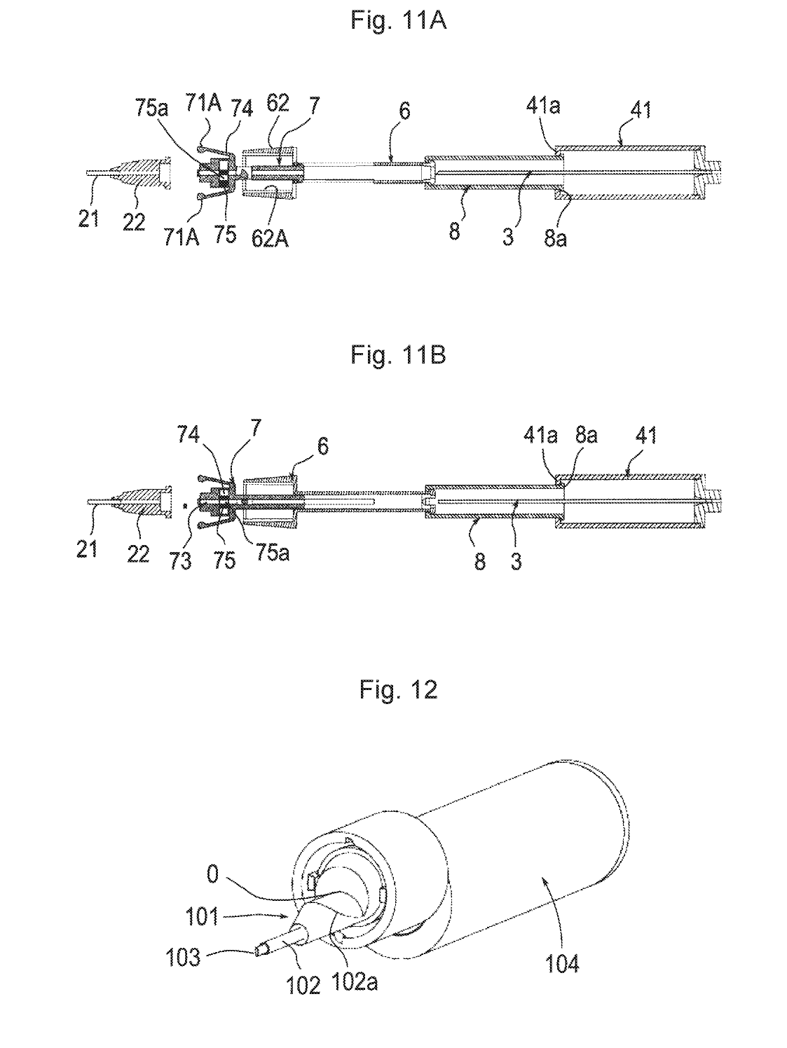

On the other hand, when the above-mentioned inner pipe 7 is pulled (advanced) from the above-mentioned outer pipe 6, the restriction by the guide grooves 62A (arm opening/closing part 62) is released as shown in FIGS. 11A and 11B so that the above-mentioned four arms 71A are unfolded by elasticity of the arm 71A itself to release the above-mentioned outer needle hub 22.

As described above, gripping means for gripping the above-mentioned outer needle hub 22 is constituted by the above-mentioned four arms 71A and the above-mentioned arm opening/closing part 62. In a situation where the above-mentioned four arms 71A are retracted into the arm opening/closing part 62, the outer needle hub 22 is grasped as shown in FIGS. 4 and 5. In a situation where the above-mentioned four arms 71A are advanced from the arm opening/closing part 62, the outer needle hub 22 is released as shown in FIGS. 11A and 11B.

In particular, the above-mentioned four arms 71A are formed to have an unfolded shape with respect to the periphery of the inner pipe 7 in advance as shown in FIG. 3.

Further, in the case where the arms 71A of the above-mentioned inner pipe 7 are inserted (accommodated) in the guide grooves 62A (see FIG. 6A) of the above-mentioned outer pipe 6, the above-mentioned four arms 71A are folded by the above-mentioned arm opening/closing part 62. That is to say, the above-mentioned arm opening/closing part 62 folds the above-mentioned four arms 71A so as to cause the above-mentioned four arms 71A to grasp the above-mentioned outer needle hub 22.

Furthermore, as the above-mentioned inner pipe 7 is pulled (advanced) from the above-mentioned outer pipe 6, the above-mentioned four arms 71A are pulled out of the above-mentioned arm opening/closing part 62 and return to the originally unfolded state as shown in FIGS. 11A and 11B. That is to say, the above-mentioned arm opening/closing part 62 causes the above-mentioned four arms 71A to be in the open state, and the above-mentioned four arms 71A allows the above-mentioned catheter 2 to be released.

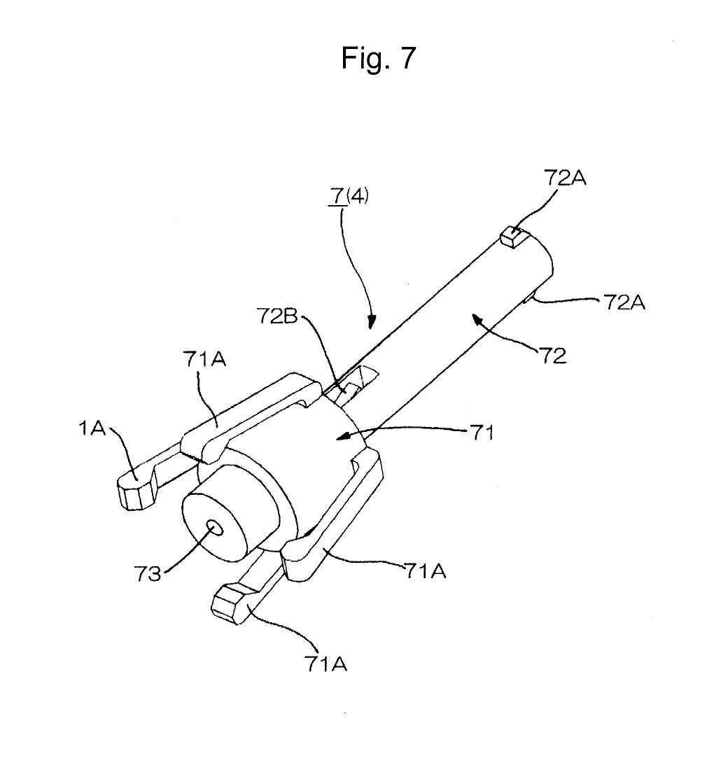

As shown in FIG. 7, the above-mentioned inner pipe 7 has a head part 71 provided with the above-mentioned four arms 71A, a shaft 72 having a diameter smaller than that of the above-mentioned head part 71, and a through hole 73 which penetrates the centers of the above-mentioned head part 71 and the above-mentioned shaft 72, and through which the above-mentioned inner needle 3 is inserted, thus being cylindrically formed as a whole.

The above-mentioned shaft 72 is provided with a projection 72A which is moveably accommodated in the above-mentioned groove 61 of the above-mentioned outer pipe 6, and a standing piece 72B which is pushed out to stand by the inner needle 3 in the above-mentioned through hole 73 and engaged with the above-mentioned groove 61.

In addition, a diameter of a through hole 73 is formed slightly larger than a diameter of the inner needle 3 so that sliding resistance at pulling operation of the inner needle 3 is reduced.

The above-mentioned projections 72A are formed axisymmetrically at two places, an upper surface and an under surface of the above-mentioned shaft 72, so as to respectively correspond to the above-mentioned grooves 61 (see FIG. 4). Further, the above-mentioned groove 61 and the above-mentioned projection 72A restrict the back and forth movement of the above-mentioned inner pipe 7 with respect to the above-mentioned outer pipe 6 (back and forth movement of the above-mentioned outer pipe 6 with respect to the inner pipe 7).

It should be noted that when the above-mentioned inner pipe 7 is advanced relatively to the above-mentioned outer pipe 6, the above-mentioned projection 72A is arranged to be engaged by an end portion 61a of the groove 61, so as not to separate the above-mentioned outer pipe 6 from the inner pipe 7.

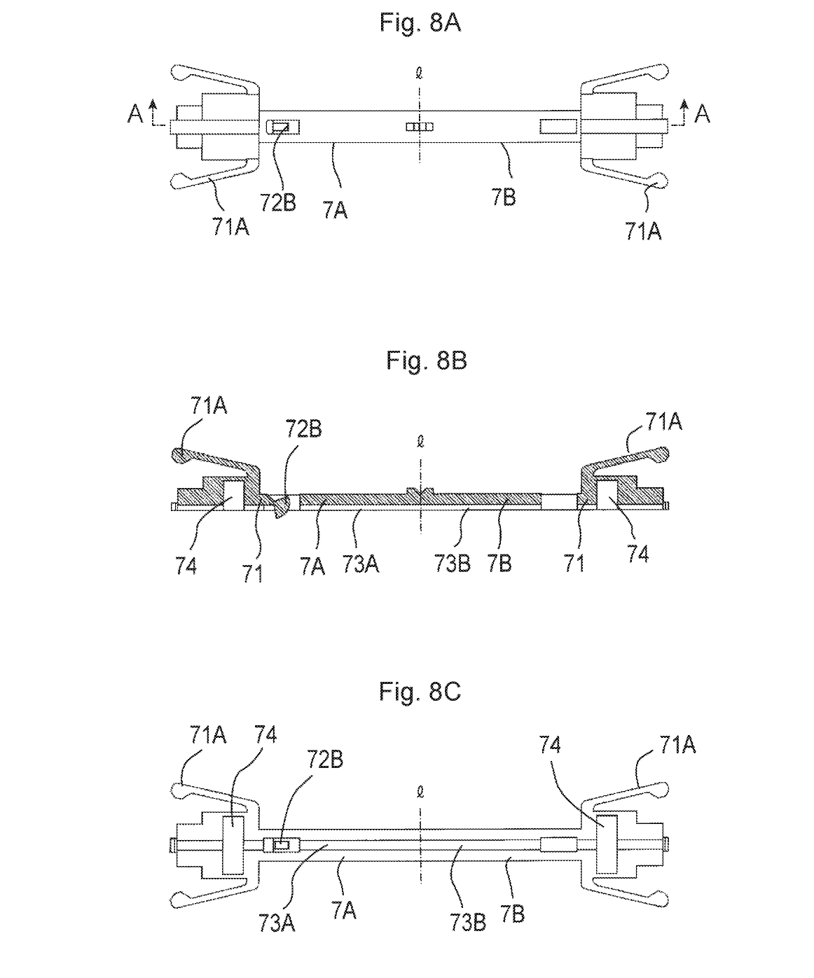

Here, as shown in FIGS. 8A, 8B and 8C, the above-mentioned inner pipe 7 is a component in which the lower part 7A (left-hand side) provided with the above-mentioned standing piece 72B and the upper part 7B (right-hand side) which are on opposite sides of a central line 1 are integrally formed. It is formed by folding the above-mentioned lower part 7A and the above-mentioned upper part 7B along a fold line (central line 1).

Further, grooves 73A and 73B whose cross sections are in the shape of a semicircle are formed along the axial direction of the above-mentioned inner pipe 7 in the centers of the above-mentioned lower part 7A and the above-mentioned upper part 7B, respectively. When the above-mentioned lower part 7A and the above-mentioned upper part 7B are folded, these grooves 73A and 73B form one through hole 73.

Furthermore, as shown in FIG. 4, as the above-mentioned inner needle 3 is inserted (accommodated) in the above-mentioned through hole 73, the above-mentioned standing piece is pushed out to stand up by the periphery of the above-mentioned inner needle 3 and engages with an end portion of the above-mentioned groove 61.

Here, the above-mentioned standing piece 72B engages with the end portion on the above-mentioned catheter 2 side of the above-mentioned groove 61, and therefore sandwiches the above-mentioned outer pipe 6 in conjunction with the above-mentioned head part 71 of the above-mentioned inner pipe 7, thus being engaged by the above-mentioned outer pipe 6.

In other words, in the case where the inner needle 3 is in the above-mentioned through hole 73, the above-mentioned inner needle 3 allows the above-mentioned standing piece 72B to stand up, the outer pipe 6 and the inner pipe 7 are unified, and the above-mentioned outer pipe 6 is inhibited from being pulled out of the above-mentioned inner pipe 7.

Further, when the above-mentioned inner needle 3 is pulled from the inside of the above-mentioned through hole 73 and the above-mentioned standing piece 72B is not pushed out by the inner needle 3, the standing piece 72B pivots in a direction of blocking the above-mentioned through hole 73 so as to be accommodated within (returns to) the above-mentioned inner pipe 7 and is disengaged from the above-mentioned groove 61.

In other words, in the case where the inner needle 3 is not in the above-mentioned through hole 73, the above-mentioned standing piece 72B is not engaged by the above-mentioned outer pipe 6, so that the outer pipe 6 and the inner pipe 7 may be separable and the above-mentioned inner pipe 7 can be pulled from the above-mentioned outer pipe 6.

In case the standing piece 72B has restoring force (elastic force), the standing piece 72B pivots in a direction of blocking the above-mentioned through hole 73 by the restoring force (elastic force) and blocks the above-mentioned through hole 73.

On the other hand, even if the standing piece 72B does not have restoring force (elastic force) or restoring force (elastic force) of the standing piece 72B is weak whereby the standing piece 72B does not pivot in a direction of blocking the above-mentioned through hole 73 by the restoring force (elastic force), when the inner pipe 7 is separated from the outer pipe 6, the standing piece 72B is raised by the end portion 61a of the groove 61, pivots in a direction of blocking the above-mentioned through hole 73, and blocks the above-mentioned through hole 73.

As described above, the standing piece 72B is raised by the end portion 61a of the groove 61, pivots in a direction of blocking the above-mentioned through hole 73 (the direction of the arrow shown in FIGS. 4 and 10A and 10B), and blocks the above-mentioned through hole 73. Therefore in case the puncture device is not used for a long time because of long storage for example, and the standing piece 72B have lost restoring force (elastic force), it is possible to block the above-mentioned through hole 73 reliably.

In addition, when a contact pressure between the standing piece 72B and the inner needle 3 is set weak (restoring force (elastic force) of the standing piece 72B is set weak), the standing piece 72B is raised by the end portion 61a of the groove 61, pivots in a direction of blocking the above-mentioned through hole 73, and blocks the above-mentioned through hole 73 as described above. Therefore it is possible to reduce sliding resistance at the pulling operation of the inner needle.

Further, as shown in FIGS. 4, 5, and 8A, 8B and 8C, a cylindrical space 74 which leads to the through hole 73 for insertion of the inner needle 3 is formed in a head part 71 of the inner pipe 7.

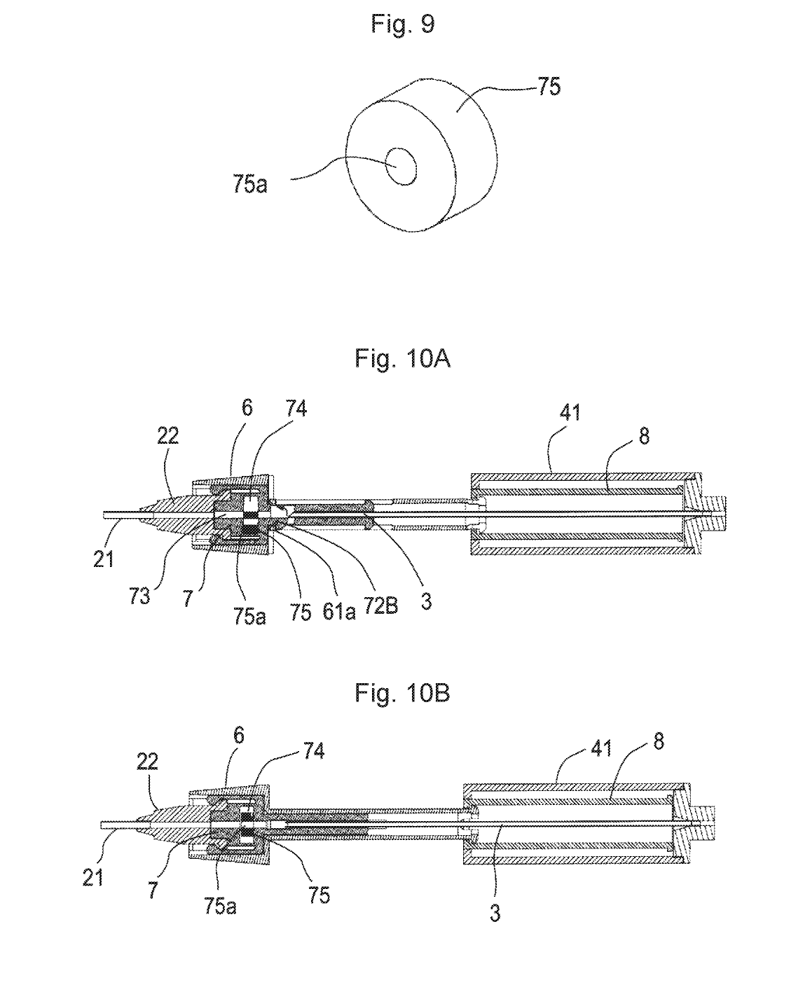

In addition, as shown in FIG. 9, a cylindrical block body 75 which is provided with a through hole 75a (the through hole 75a constitutes at least a part of the through hole 73) for insertion of the inner needle 3 is accommodated in the space 74. The space 74 is formed larger than an outer diameter of the block body 75 so that the block body can move in the space 74.

The space 74 and the block body 75 constitute a through hole block means for blocking communicated condition of the through hole 73, which makes at least part of the through hole 73 move from a position where the inner needle 3 is inserted to a position where the inner needle 3 is blocked.

As described above, since the block body 75 is placed in the space 74 in the head part 71 of the inner pipe 7, once a tip of the inner needle 3 is pulled out through the through hole 75a of the block body 75, the block body 75 falls in the space 74 by the block body's own weight. This will cause the through hole 75a to be placed elsewhere than on the line that extends from the through hole 73 and a communication state of the through hole 73 to be blocked.

In addition, since a diameter of the through hole 75a is formed slightly larger than that of the inner needle, it is possible to reduce the sliding resistance at the pulling operation of the inner needle 3.

Further, as shown in FIGS. 4 and 5, it is arranged that the relay pipe 8 which extends the outer pipe 6 is provided between the above-mentioned inner needle hub 41 and the outer pipe 6. The relay pipe 8 is not necessary, and as shown in FIG. 3, the relay pipe 8 may not be used.

For example, flexibility is given to the relay pipe 8 by a soft synthetic resin material for forming the relay pipe 8, even if a direction to pull the inner needle hub 41 is an inclined direction (which is not on an extension of the inner needle 3) and bending force is applied to the relay pipe 8, it is possible to prevent the relay pipe 8 from being damaged by buckling etc.

This relay pipe 8, formed in the shape of a cylinder, has an outer diameter allowing itself to be accommodated inside the above-mentioned inner needle hub 41, and has an inner diameter allowing the outer pipe 6 to be accommodated in itself. It is arranged that an engaging portion 8a is formed at one end of this relay pipe 8 and engages with a projection formed at the end portion 63 of the outer pipe 6. Further, it is arranged that a projection 8b is formed at the other end of the relay pipe 8 and engaged with an engaging portion formed at the end portion 41a of the inner needle hub 41.

In the case where the relay pipe 8 is not used, it is arranged that an engaging portion formed at end portions 41a of the above-mentioned inner needle hub 41 fits an engaging portion formed at end portions 63 of the above-mentioned outer pipe 6 so as not to be separated (spaced apart) from each other.

Next, a case where such a puncture device 1 is used will be described. It should be noted that below explanation has been described with reference to the case where restoring force (elastic force) of a standing piece 72B is weak and the standing piece 72B is not in pressure contact with an inner needle.

Firstly, the protector 5 is removed from the puncture device 1 shown in FIG. 1, and the catheter 2 and the inner needle 3 are exposed as shown in FIGS. 4 and 5. Further, the above-mentioned outer needle 21 and the above-mentioned inner needle 3 are punctured to a blood vessel (patient's body 110).

Then, in order to indwell the above-mentioned outer needle 21, the above-mentioned inner needle hub 41 is moved along the axial direction shown by the arrows in FIGS. 4 and 5 in a direction away from the above-mentioned outer needle 21 (pull-out operation is performed). The above-mentioned syringe 4 is expanded by pulling out this inner needle hub 41. In particular, in a situation where the above-mentioned outer needle 21 is indwelled, when the above-mentioned inner needle hub 41 is moved along the axial direction away from the above-mentioned outer needle 21, the above-mentioned inner needle hub 41 moves along the axial direction, and the above-mentioned syringe 4 is expanded as a whole. At this time, the outer needle hub 22 is retained by the inner pipe 7, and the standing piece 72B unifies the inner pipe 7 with the outer pipe 6.

Therefore, as the inner needle hub 41 for retaining the rear end portion (base portion) of the inner needle 3 is moved along the axial direction, the inner needle 3 similarly moves along the axial direction. Further, the inner needle 3 is pulled from the outer needle 21, and the thus pulled inner needle 3 is covered with the inner pipe 7, the outer pipe 6, and the inner needle hub 41 (see FIGS. 10A and 10B).

In the pull-out operation of the inner needle hub, once a tip portion of the inner needle 3 passes through a through hole 75a of a block body 75 and the inner needle is pulled out from the block body 75, the block body moves down in the space 74 by the block body's own weight. This will cause the through hole 75a to be placed elsewhere than on the line that extends from the through hole 73 and a communication state of the through hole 73 to be blocked (see FIGS. 10A and 10B).

Therefore, after the through hole 73 is blocked by the block body 75, even if the force of moving the inner needle hub 41 to the catheter 2 side is applied, the block body 75 inhibits the movement of the inner needle 3 and the inner needle 3 does not return into the outer needle 21 gain.

Further, the above-mentioned inner needle hub 41 is moved along the axial direction away from the above-mentioned outer needle 21. As shown in FIGS. 10A and 10B, after the tip of the inner needle 3 passes over the standing piece 72B, the standing piece 72B is free from a force by a side surface of the inner needle 3. At this time, the standing piece 72B does not pivot in a direction of blocking the above-mentioned through hole 73 because of weak restoring force (elastic force) of the standing piece 72B so, a locking state between the standing piece 72B and the outer pipe 6 is maintained.

Then, the relay pipe 8 is pulled out of the inner needle hub 41, and a fitting portion 8b of the relay pipe 8 fits a fitting portion formed at the end portion 41a of the inner needle hub to be full extension. Then, the standing piece 72B is raised up by an end portion 61a of a groove 61 of the outer pipe 6. The raised standing piece 72B pivots in a direction of blocking the above-mentioned through hole 73 (the direction of the arrow shown in FIGS. 10A and 10B) and blocks the through hole 73 with disengaging the above-mentioned outer pipe 6.

As a result, the outer pipe 6 and the inner pipe 7 become separable, and it follows that as the above-mentioned inner needle hub 41 moves, the above-mentioned outer pipe 6 moves along the axial direction.

Since the above-mentioned groove 61 is guided by the above-mentioned projection 72A, the above-mentioned inner pipe 7 is pulled from the above-mentioned outer pipe 6, whilst being guided by the projection 72A of the above-mentioned inner pipe 7.

In addition, after the standing piece 72B is raised up, even if the force of moving the inner needle hub 41 to the catheter 2 side is applied, the above-mentioned standing piece 72B inhibits the movement of the inner needle 3, which does not return into the outer needle 21 again.

As described above, after the standing piece 72B is raised up, the above-mentioned standing piece 72B inhibits the movement of the inner needle 3, and moreover, the block body 75 inhibits the movement of the inner needle 3. Therefore, it is possible to inhibit surely the inner needle return into the outer needle 21 again.

Further, when the above-mentioned inner needle hub 41 is moved away from the above-mentioned outer needle 21 along the axial direction, the above-mentioned outer pipe 6 is moved, and the above-mentioned inner pipe 7 is pulled from the above-mentioned outer pipe 6, then the above-mentioned four arms 71A are opened as shown in FIGS. 11A and 11B, and the retention of the outer needle hub 22 by the above-mentioned four arms 71A is released.

That is to say, by separating the above-mentioned inner needle hub 41 from the above-mentioned outer needle 21 along the axial direction and expanding the above-mentioned syringe 4, the above-mentioned inner needle 3 pulled from the above-mentioned catheter 2 is accommodated inside the above-mentioned outer pipe 6, and the inner needle hub 41, then the outer needle hub 22 is released from the state where the outer needle hub 22 is retained by the above-mentioned four arms 71A and arm opening/closing parts 62.

Accordingly, whilst the above-mentioned catheter 2 (outer needle 21) indwelling in a blood vessel, the above-mentioned inner needle 3 is pulled from the above-mentioned outer needle 21 and accommodated inside the above-mentioned inner needle hub 4, and the above-mentioned outer needle hub 22 is removed from the above-mentioned syringe 4.

Thus, only by pulling the above-mentioned inner needle 3 from the above-mentioned outer needle 21, the above-mentioned puncture device 1 can accommodate the above-mentioned inner needle 3 in the above-mentioned inner needle hub 41, and the above-mentioned outer needle hub 22 can be removed from the above-mentioned inner needle hub 4.

In addition, although the above mentioned use of the puncture device has been described with reference to the case where the restoring force (elastic force) of the standing piece 72B is weak and the standing piece 72B is not in pressure contact with an inner needle, the restoring force (elastic force) may be applied to the standing piece 72B without increasing a slide resistance at the pulling operation of the inner needle 3 too far.

It should be noted that although the first preferred embodiment has been described with reference to the case where the block body 75 and the space 74 are described as a cylindrical shape, the present invention is not particularly limited to this structure. For example, the block body 75 may be formed in a globe shape and the space 74 may be formed in a rectangular shape. It is only needed that the block body 75 is formed in a movable state in the space 74.

Next, a second preferred embodiment will be described with reference to FIGS. 12 to 20. A through hole block means in this embodiment is arranged that the inner pipe and the outer pipe relatively rotate when the outer pipe moves relative to the inner pipe, and has a characteristic feature that the through hole of the inner needle moves from a position where the inner needle is inserted to a position where the inner needle is blocked and insertion of the inner needle is blocked.

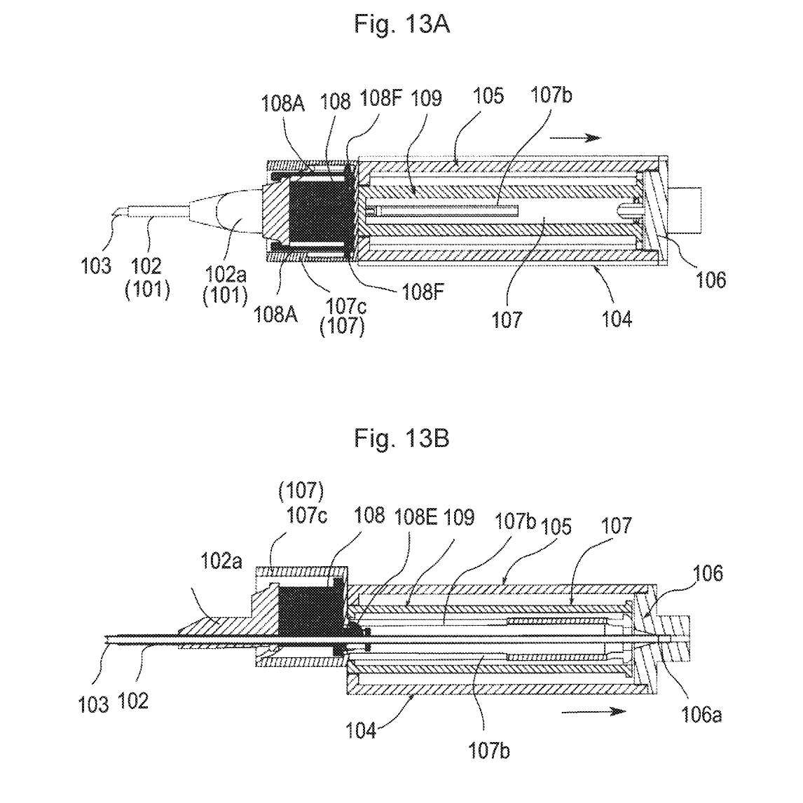

As shown in FIGS. 12 and 13A and 13B, a puncture device 100 is provided with an outer needle 102, an inner needle 103 whose tip portion (left-hand side) is inserted in the above-mentioned outer needle 102, a cylindrical syringe 104 for retaining an end portion (base portion) of the above-mentioned inner needle 103, and a protector (not shown in the figures) which covers the above-mentioned outer needle 102 and the above-mentioned inner needle 103. Further, in the above-mentioned puncture device 100, all the components except the above-mentioned inner needle 103 are made of resin.

Further, as shown in FIGS. 12 and 13A and 13B, the above-mentioned catheter 101 has the outer needle 102 formed of a flexible hollow pipe and the outer needle hub 102a for retaining the base portion of the outer needle 102.

A position of the outer needle 102 that is arranged in the outer needle hub 102a is placed at a position deviated from the center position O of the outer needle hub 102a as shown in FIGS. 12 and 13B.

As shown in FIG. 13B, the above-mentioned syringe 104 is provided with a cylindrical inner needle hub 105 and a plug 106 which is press fitted into and attached to a base portion (right-hand side) of the above-mentioned inner needle hub 105 and has a substantially cylindrical needle retaining part 106a by which the base portion of the above-mentioned inner needle 103 is retained.

Further, the above-mentioned syringe 104 is provided with an outer pipe 107 fitted inside the above-mentioned inner needle hub 105 so as to be moveable to and fro, and an inner pipe 108 having two arms 108A for gripping the above-mentioned outer needle hub and fitted inside the above-mentioned outer pipe 106 so as to be freely moveable.

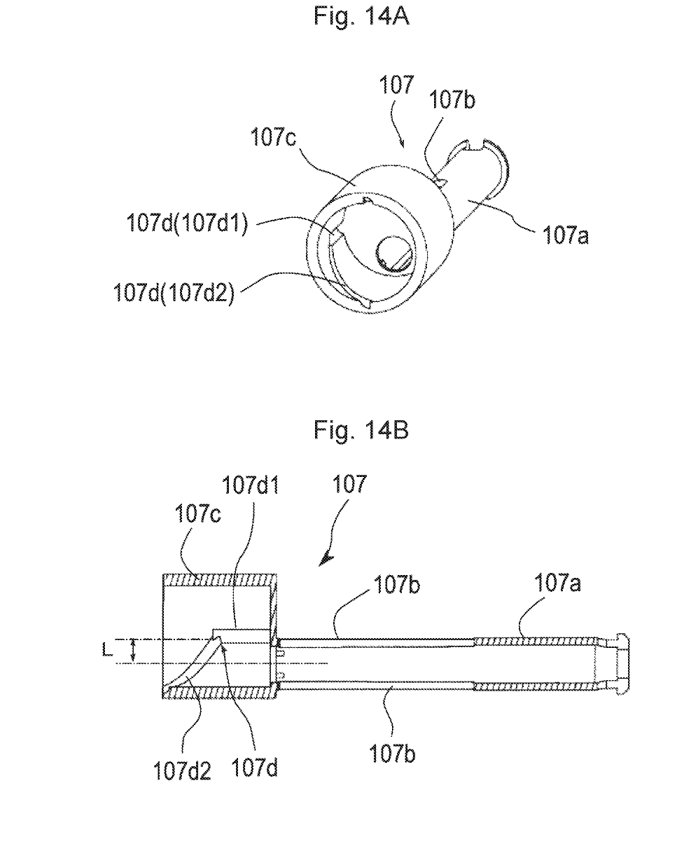

As shown in FIGS. 14A and 14B, the above-mentioned outer pipe 107 is provided with a groove 107b formed along an axis of a shaft 107a of the above-mentioned outer pipe 107 (formed along a direction of back and forth movement of the inner pipe 7) and an arm opening/closing part 107c which accommodates the above-mentioned two arms 108A, and cylindrically formed as a whole.

The center position of the shaft 107a is arranged at a position L deviated from the center line of the arm opening/closing part 107c as shown in FIG. 14B. That is to say, the shaft 107a is arranged at a downward position in the arm opening/closing part 107c and the center line of the arm opening/closing part 107c does not coincide with an axis of a shaft 107a.

As shown in FIG. 14B, the above-mentioned grooves 107b are axisymmetrically formed at two places, an upper part and a lower part in the periphery of the above-mentioned outer pipe 107.

Further, as shown in FIG. 13A, when the above-mentioned inner pipe 108 is accommodated in the above-mentioned outer pipe 107, the above-mentioned two arms 107A are folded, and the above-mentioned arm opening/closing part 107c grasps the above-mentioned outer needle hub 102a.

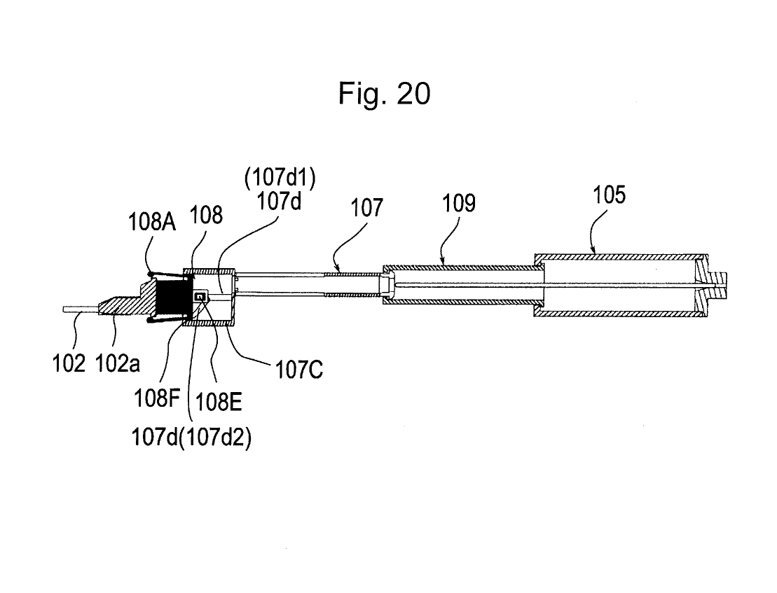

On the other hand, when the above-mentioned inner pipe 108 is pulled (advanced) from the above-mentioned outer pipe 107, the restriction by the arm opening/closing part 107c is released as shown in FIG. 20 so that the arms 108A are unfolded by elasticity of the arm 108A itself to release the above-mentioned outer needle hub 102a.

Grooves 107d for movably attaching projecting parts 108F formed on an outer surface of the inner pipe 108 are formed on an inner periphery of the arm opening/closing part 107c.

The grooves 107d are formed by arranging right and left sides in a pair. A linear portion 107d1 in which the groove is formed linearly is formed at a side of the shaft 107a and next to the linear portion 107d1, a spiral portion 107d2 in which the groove is formed spirally is formed.

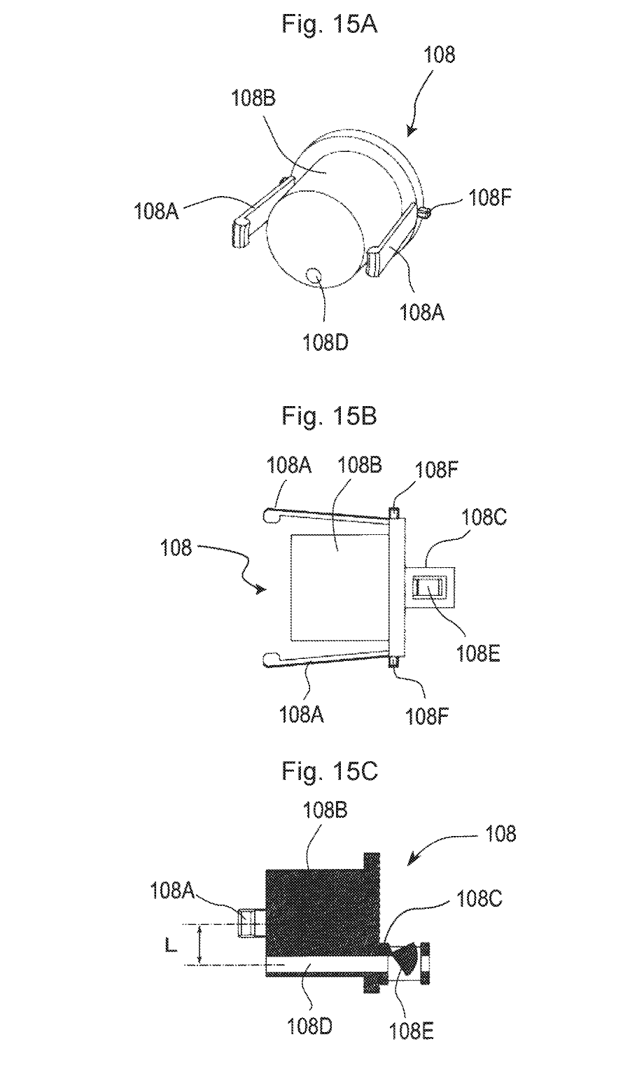

As shown in FIGS. 15A, 15B, and 15C, the above-mentioned inner pipe 108 has a head part 108B provided with the above-mentioned two arms 108A, a shaft 108C having a diameter smaller than that of the above-mentioned head part 108B, and a through hole 108D which penetrates the above-mentioned head part 108B and the above-mentioned shaft 108C, and through which the above-mentioned inner needle 3 is inserted.

As shown in FIG. 15C, the shaft 108C is arranged at a position deviated from the center line of the head part 108B and the center line of the head part 108B does not coincide with an axis of a shaft 108C.

According to the above, the through hole 108D formed on the inner pipe 108 is arranged at a position L which is away from (deviated from) the central axis of the inner pipe 108.

It is arranged that, when the head part 108B of the inner pipe 108 is accommodated in the arm opening/closing part 107c of the outer pipe 107, the above-mentioned shaft 108C is accommodated in the shaft 107a of the outer pipe 107.

The above-mentioned shaft 108C is provided with a standing piece 108E which is pushed out to stand by the inner needle 103 in the above-mentioned through hole 108D and engaged with the above-mentioned slit 107b.

The standing piece 108E is same with the standing piece 72B in the first preferred embodiment, and will not be described further in detail. In addition, a diameter of the through hole 108D is formed slightly larger than a diameter of the inner needle 3 so that sliding resistance at pulling operation of the inner needle 3 is reduced.

A pair of projecting part 108F is formed on an outer surface of an edge portion at a shaft 108C side of the head part 108B. As described above, the pair of projecting part 108F is formed axisymmetrically each other so that the projecting parts 108F respectively corresponds to the above-mentioned grooves 107d (See FIGS. 14A and 14B).

The inner pipe 108 and the outer pipe 107 relatively rotate since the pair of projecting part 108F moves along the grooves 107d when the inner pipe 108 moves relative to the outer pipe 107.

According to the above, since the through hole 108D formed on the inner pipe 108 is arranged at a position L which is away from the central axis of the inner pipe 108 and the through hole block means includes the grooves 107d which contain the spiral portion formed on the inner surface of the outer pipe 107 and projecting parts 108F formed on the outer surface of the inner pipe 108, which is movable in the grooves 107d, the inner pipe 108 and the outer pipe 107 relatively rotate whereby it is possible to move the through hole 108D formed on the inner pipe 108 from a position where the inner needle is inserted to a position where the inner needle is blocked.

In addition, after the pair of projecting part 108F moves along the grooves 107d and the arms 108A are unfolded, since the pair of projecting part 108F is locked at end portion of the spiral portion 107d2 in the groove 107d on the outer pipe 107, relative movement between the outer pipe 107 and the inner pipe 108 is regulated. That is to say, the outer pipe 107 and inner pipe 108 are configured so as not to separate from each other.

Further, as shown in FIGS. 13A and 13B, it is arranged that the relay pipe 109 which extends the outer pipe 107 is provided between the above-mentioned inner needle hub 105 and the outer pipe 107. Since the relay pipe 109 is used in the same way as the first embodiment, the relay pipe 109 will not be described further in detail.

Next, a case where such a puncture device 100 is used will be described. It should be noted that below explanation has been described with reference to the case where restoring force (elastic force) of a standing piece 102E is weak and the standing piece 102E is not in pressure contact with an inner needle 103 in the same way as the first embodiment.

Firstly, the protector is removed from the puncture device 100, and the catheter 101 and the inner needle 103 are exposed as shown in FIGS. 13A and 13B. Further, the above-mentioned outer needle 102 and the above-mentioned inner needle 103 are punctured to a blood vessel (patient's body).

Then, in order to indwell the above-mentioned outer needle 102, the above-mentioned inner needle hub 105 is moved along the axial direction and in a direction shown by the arrows in FIGS. 13A and 13B away from the above-mentioned outer needle 102 (pull-out operation is performed). The above-mentioned syringe 104 is expanded by pulling out this inner needle hub 105.

In particular, in a situation where the above-mentioned outer needle 102 is indwelled, when the above-mentioned inner needle hub 105 is moved along the axial direction away from the above-mentioned outer needle 102, the above-mentioned inner needle hub 105 moves along the axial direction, and the above-mentioned syringe 4 is expanded as a whole. At this time, the outer needle hub 105 is retained by the inner pipe 108, and the standing piece 108E unifies the inner pipe 108 with the outer pipe 107.

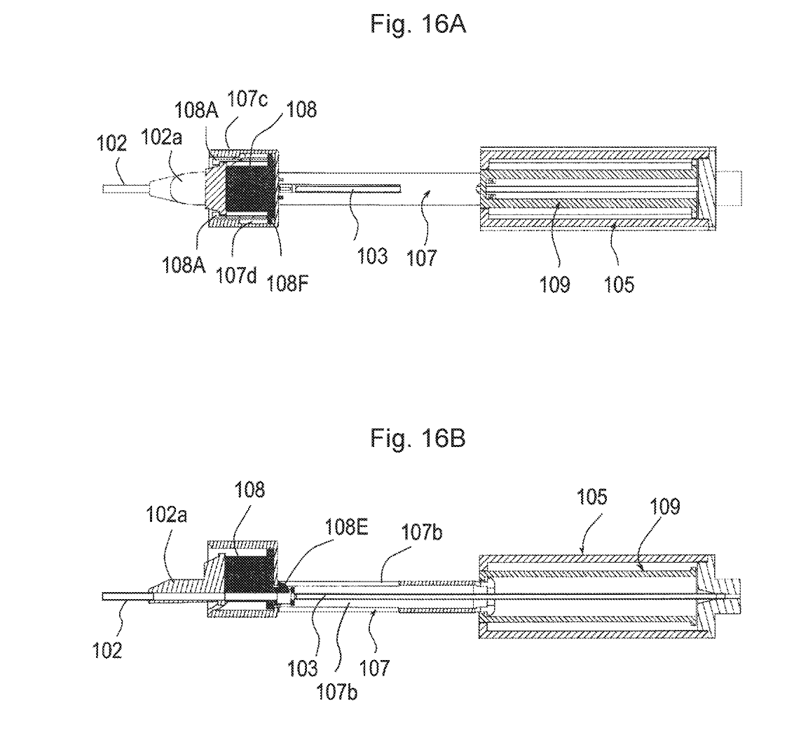

Therefore, as the inner needle hub 105 for retaining the rear end portion (base portion) of the inner needle 103 is moved along the axial direction, the inner needle 103 similarly moves along the axial direction. Further, the inner needle 103 is pulled from the outer needle 102, and the thus pulled inner needle 3 is covered with the outer pipe 107, and the inner needle hub 105 (see FIGS. 16A and 16B).

The above-mentioned inner needle hub 105 is further moved away from the above-mentioned outer needle 102 along the axial direction. Then, when the tip of the inner needle 103 passes by the standing piece 108E as shown in FIGS. 16A and 16B, the force is not applied to the standing piece 108E from the side of the above-mentioned inner needle 103. At this time, the standing piece 108E does not pivot in a direction of blocking the above-mentioned through hole 108D because of weak restoring force (elastic force) of the standing piece 108E so, a locking state between the standing piece 108E and the outer pipe 107 is maintained.

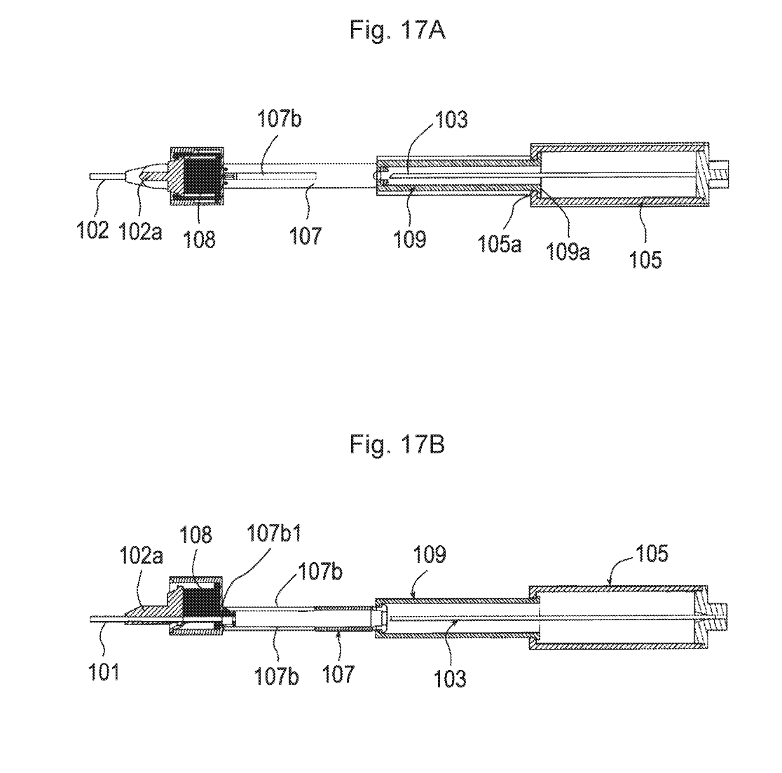

Then, as shown in FIGS. 17A and 17B the relay pipe 109 is pulled out of the inner needle hub 105, and a fitting portion 109a of the relay pipe 109 fits a fitting portion formed at the end portion 105a of the inner needle hub to be full extension. Then, the standing piece 108E is raised up by an end portion 107b1 of a groove 107b of the outer pipe 107. The raised standing piece 108E pivots in a direction of blocking the above-mentioned through hole 108D and blocks the through hole 108D with disengaging the above-mentioned outer pipe 107.

As a result, the outer pipe 107 and the inner pipe 108 become separable, and it follows that as the above-mentioned inner needle hub 105 moves, the above-mentioned outer pipe 107 moves along the axial direction.

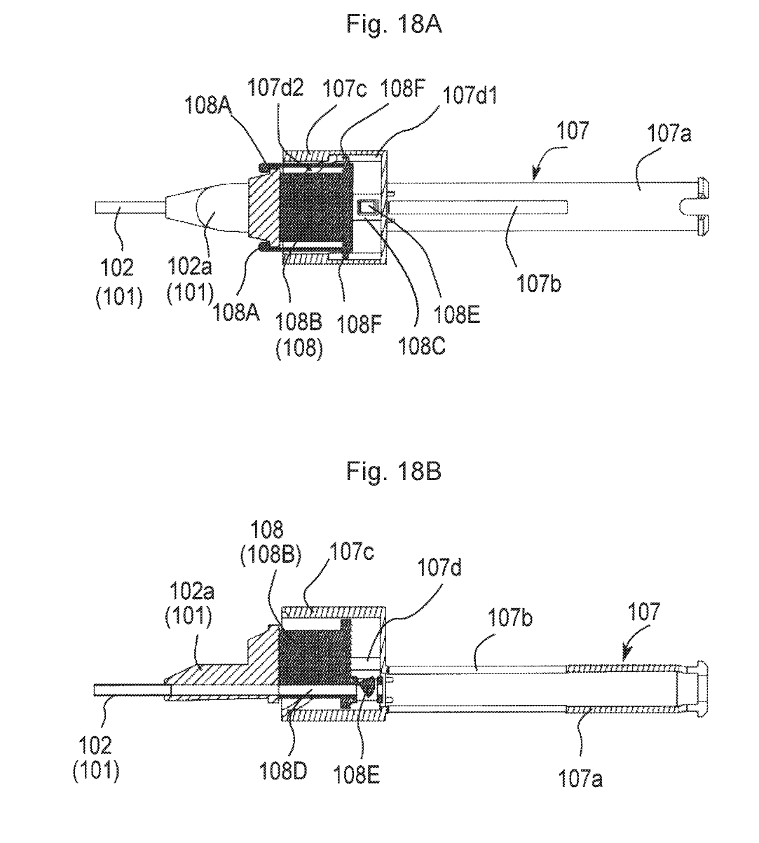

In addition, after the standing piece 108E is raised up, as shown in FIG. 18B, even if the force of moving the inner needle hub 105 to the catheter 101 side is applied, the above-mentioned standing piece 108E inhibits the movement of the inner needle 103, which does not return into the outer needle 102 again.

Once the locking state between the outer pipe 107 and the inner pipe is released, as shown in FIGS. 18A and 18B, projections 108F of the inner pipe 108 are guided by the linear portion 107d1 of the grooves 107d and move.

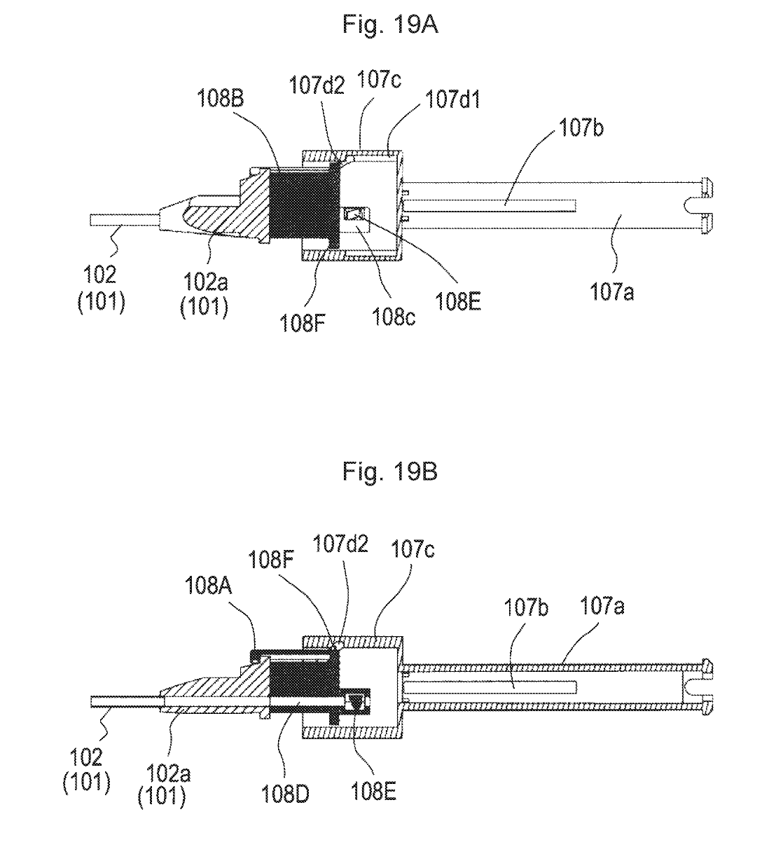

Subsequently, while projections 108F of the inner pipe 108 are, as shown in FIGS. 19A and 19B, guided by the spiral portion 107d2 of the grooves 107d and move, the outer pipe 107 rotates relative to the inner pipe. In actuality, the outer pipe 107 moves and rotates relative to the inner pipe 108 which grasps a fixed catheter 101.

As a result, the through hole 108D of the inner pipe is moved from a position where the inner needle is inserted to a position where the inner needle is blocked whereby a communication state of the through hole 108D is blocked.

Furthermore, the outer pipe 107 is moved as the outer pipe 107 rotates relative to the inner pipe, and when the inner pipe 108 is moved away from the outer pipe 107, as shown in FIG. 20, the above mentioned two arms 108A are opened and the retention of the outer needle hub 102a by the above-mentioned arms 108A is released. That is to say, by separating the above-mentioned inner needle hub 105 from the above-mentioned outer needle 102 along the axial direction and expanding the above-mentioned syringe 104, the above-mentioned inner needle 103 pulled from the above-mentioned catheter 101 is accommodated inside the above-mentioned outer pipe 107, and the inner needle hub 105 (sometimes the relay pipe 109), then the outer needle hub 102a is released from the state where the outer needle hub 102a is retained by the above-mentioned four arms 108A and arm opening/closing parts 107c.

Accordingly, whilst the above-mentioned catheter 101 (outer needle 102) indwelling in a blood vessel, the above-mentioned inner needle 103 is pulled from the above-mentioned outer needle 102 and accommodated inside the above-mentioned inner needle hub 105, and the above-mentioned outer needle hub 102a is removed from the above-mentioned syringe 104.

Thus, only by pulling the above-mentioned inner needle 103 from the above-mentioned outer needle 102, the above-mentioned puncture device 100 can accommodate the above-mentioned inner needle 103 in the above-mentioned inner needle hub 105, and the above-mentioned outer needle hub 102a can be removed from the above-mentioned inner needle hub 105.

As described above, after the standing piece 108E is raised up, even if the force of moving the inner needle hub 105 to the catheter 101 side is applied, the above-mentioned standing piece 108E inhibits the movement of the inner needle 103, which does not return into the outer needle 102 again.

Further, since the inner pipe 108 and the outer pipe 107 relatively rotate and the through hole 108D formed on the inner pipe 108 moves from a position where the inner needle 103 is inserted to a position where the inner needle 103 is blocked, the through hole 108D is blocked and the inner needle does not return into the outer needle 102 again.

Therefore, it is possible to surely prevent the inner needle returning into the outer needle 102 again because of a movement of the standing piece 108E and the through hole 108D of the inner pipe 108.

In addition, although the above mentioned second embodiment has been described with reference to the case where the grooves 107d are formed on the inner surface of the outer pipe 107 and the projecting parts 108F formed on the inner pipe 108, grooves may be formed on the inner pipe and projecting parts may be formed on the outer pipe to the contrary.

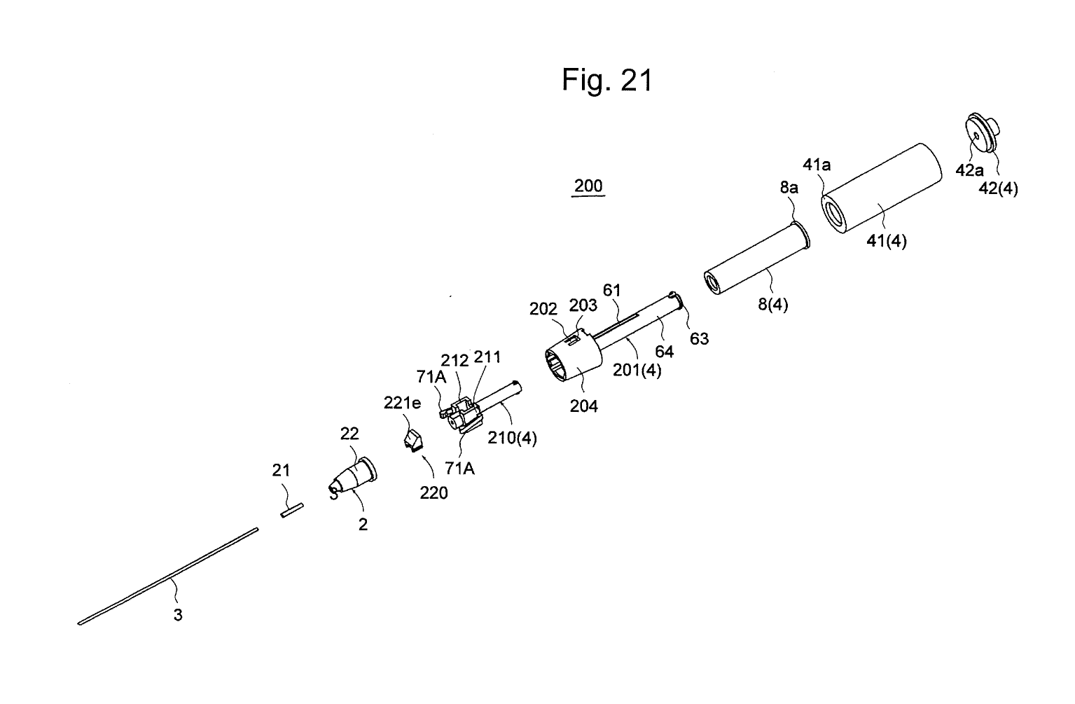

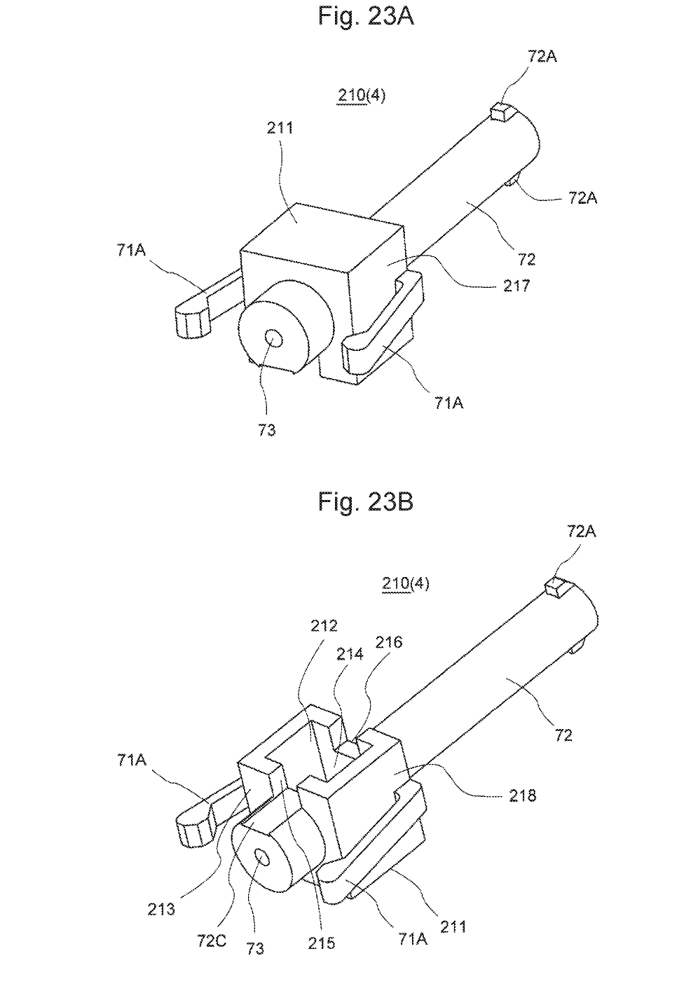

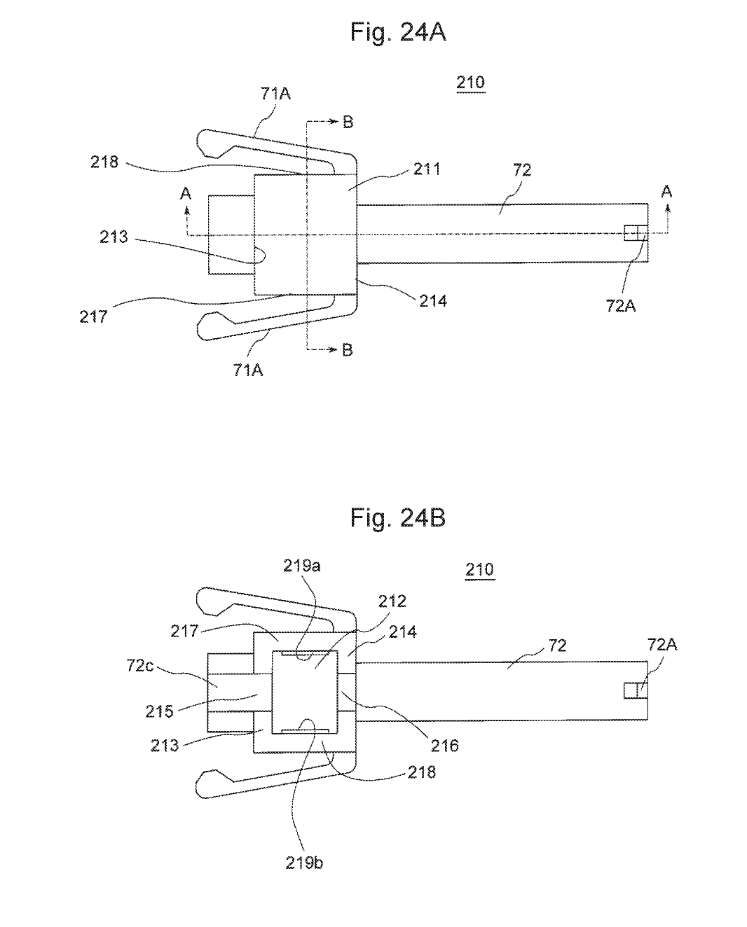

Next, a third preferred embodiment will be described with reference to FIGS. 21 to 29. A through hole block means in this embodiment includes a block body on which a through hole for inserting the inner needle is formed, a space, formed in the inner pipe, in which the block body is movably accommodated, and is arranged that when a tip of a inner needle is pulled out through the block body and an inner pipe moves with respect to an outer pipe, the block body is moved in the space by a projection formed on an inner surface of the outer pipe and an insertion of the inner needle (a through hole for insertion of the inner needle) is blocked. It should be noted that the same parts or corresponding parts are designated by the same reference signs as in the first preferred embodiment, and will not be described further in detail.

A puncture device 200 of the third embodiment differs in an outer pipe 201, an inner pipe 210, and a block body 220 from the outer pipe, the inner pipe and the block body 7 of the first embodiment.

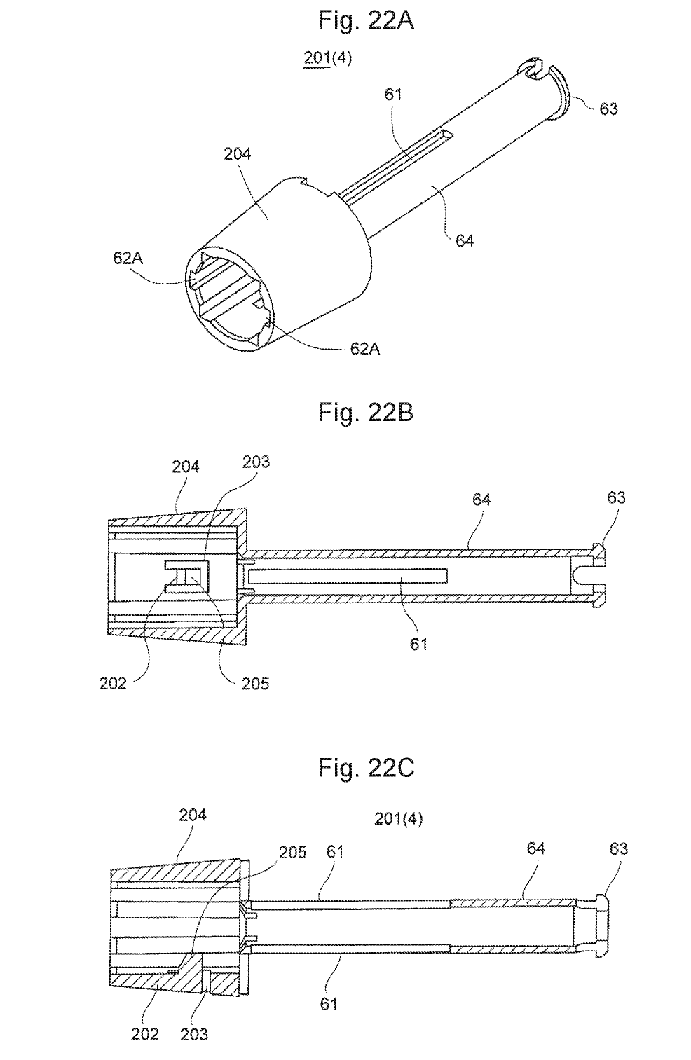

That is to say, as shown in FIGS. 21 and 22A, 22B and 22C, a tongue-like part 202 surrounded by a U-shaped through hole 203 in planar view is formed at an arm opening/closing part 204 of the outer pipe 201, which accommodates two arms 71A. A projection 205 is formed at a tip portion of the tongue-like part 202. In addition, the tongue-like part 202 is arranged that a base end portion of the tongue-like part 202 is located on a tip portion side of the outer pipe 201.

The projection 205 formed at the tip portion of the tongue-like part 202 contacts to the block body and moves the block body upward when the inner pipe 210 moves in the outer pipe 201.

Next, the inner pipe 210 is explained below.

As shown in FIGS. 21, 23A, 23B, 24A, 24B, 25A and 25B, the inner pipe 210 has a rectangular head part 211 provided with the above-mentioned two arms 71A, a shaft 72 having a diameter smaller than that of the above-mentioned head part 211, and a through hole 73 which penetrates the centers of the above-mentioned head part 211 and the above-mentioned shaft 72, and through which the above-mentioned inner needle 3 is inserted.

A concave space 212 which has a one open surface for connecting to a through hole 73 through which the above-mentioned inner needle 3 is inserted is formed on the head part 212 of the inner pipe 210.

In addition, cutout portions 215, 216 which are open on an open surface side of the concave space 212 are formed on a front wall 213 and a back wall 214 of the above mentioned head part 211.

Further, a planar portion 72C is formed on a cutout portion 215 side on the shaft 72 which is formed at a tip portion side of the head part 211.

In addition, the space 212 is for housing the block body 220. The space 212 is formed larger than an outside diameter of the block body 220 so that the block body can move in the space 212.

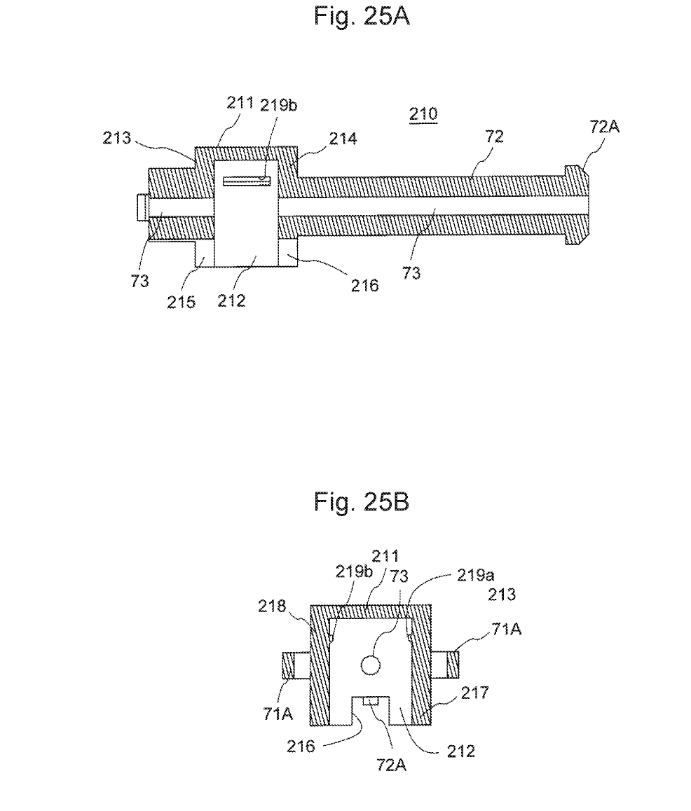

Further, projections 219a, 219b protruding into a space 212 side are formed inside of a sidewall 217 and a side wall 218 of the head part 211. As shown in FIGS. 25A and 25B, the projections 219a, 219b are arranged at a back side than the through hole 73 as seen from the open surface side of the concave space 212 and in parallel with an axis of the through hole 73.

The projections 219a, 219b are for engaging with the block body after movement of the block body.

Next, the block body 220 is explained below.

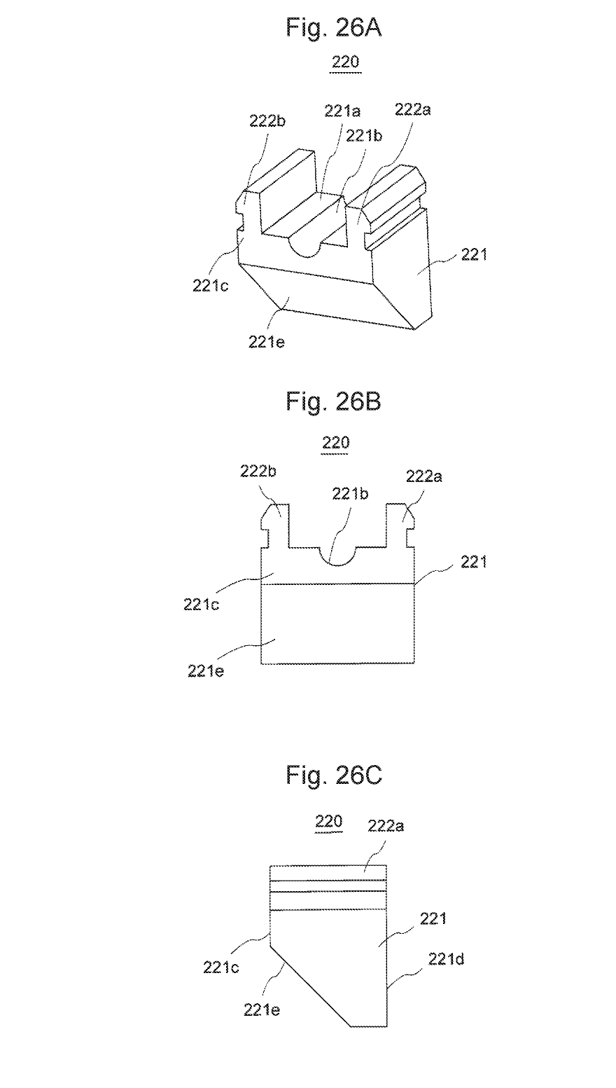

As shown in FIGS. 26A, 26B and 26C, regarding the block body 220, a semicylindrial concave portion 221b is formed on a top face of a main body 221, and hook portions 222a, 222b which extend upward from the top face 221a are formed.

The concave portion 221b is formed on the main body 221 in a front-rear direction. When the block body 220 is accommodated in the space 212, it is arranged that the inner needle 3 contacts with the block body 220 and slides inside the concave portion 221b.

In addition, when the block body 220 moves in the space 212, the hook portions 222a, 222b are engaged on the projections 219a, 219b so that the block body 220 is maintained in the space 212, and the hook portions 222a, 222b limit the movement of the block body 220.

An inclined plane 221e which is inclined so as to become downward as it goes from a front face 221c side to a rear face 221d side is formed on the front face 221c of the main body 221.

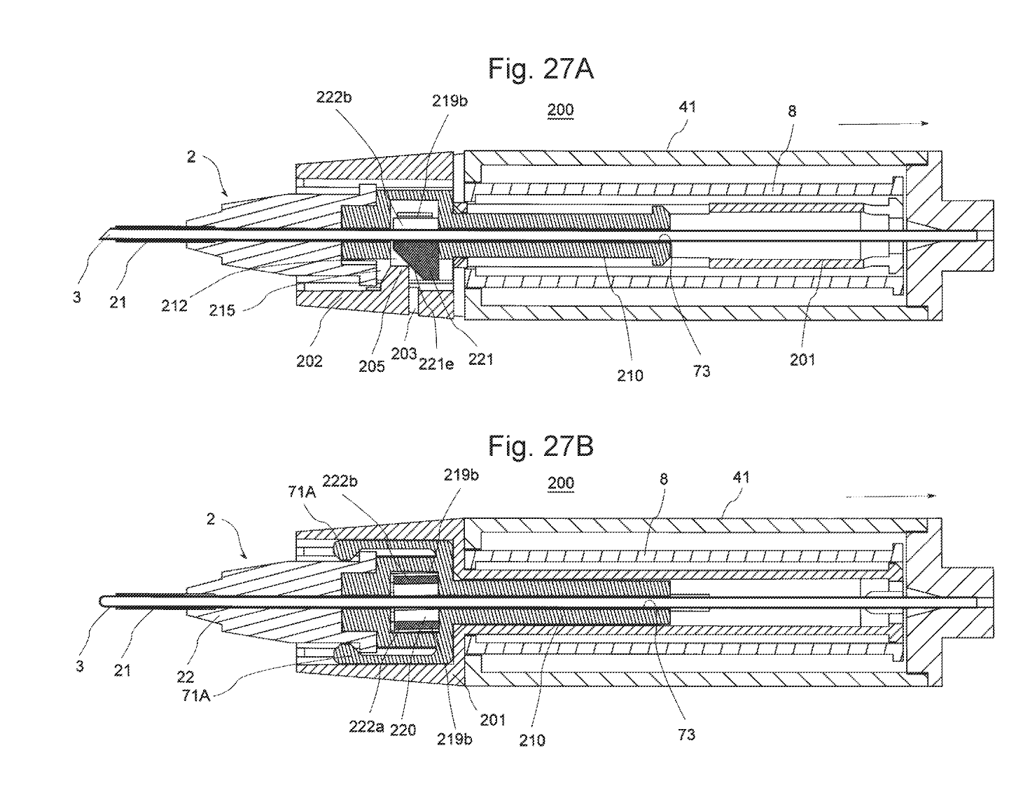

The inclined plane 221e of the main body 221 is, as shown in FIG. 27A, is accommodated in the space 212 so as to contact with a projection 205 at the tip portion of the tongue-like part 202. That is to say, the projection 205 enters the space 212 from the cutout portion 215 and comes in contact with the inclined plane 221e.

The through hole block means includes the space 212, the block body 220, and the outer pipe 201. When the outer pipe 201 moves relative to the inner pipe 210, the block body 220 is pushed up (moved) in the space 212 by the projection 205 whereby a state where the inner needle 3 is inserted is changed to a state where the inner needle 3 is blocked and a communication state of the through hole 73 is blocked.

Work and operation of the puncture device 200 in accordance with the above structured third preferred embodiment will be described with reference to FIGS. 27 to 29.

Firstly, the protector 5 is removed from the puncture device 200, and the catheter 2 and the inner needle 3 are exposed as shown in FIGS. 27A and 27B. Further, the above-mentioned outer needle 21 and the above-mentioned inner needle 3 are punctured to a blood vessel (patient's body).

Then, in order to indwell the above-mentioned outer needle 21, the above-mentioned inner needle hub 41 is moved along the axial direction shown by the arrows in FIGS. 27A and 27B and in a direction away from the above-mentioned outer needle 21 (pull-out operation is performed). As the inner needle hub 41 for retaining the rear end portion (base portion) of the inner needle 3 is moved along the axial direction, the inner needle 3 similarly moves along the axial direction. Further, the inner needle 3 is pulled from the outer needle 21, and the thus pulled inner needle 3 is covered with the inner pipe 210, the outer pipe 201, and the inner needle hub 41.

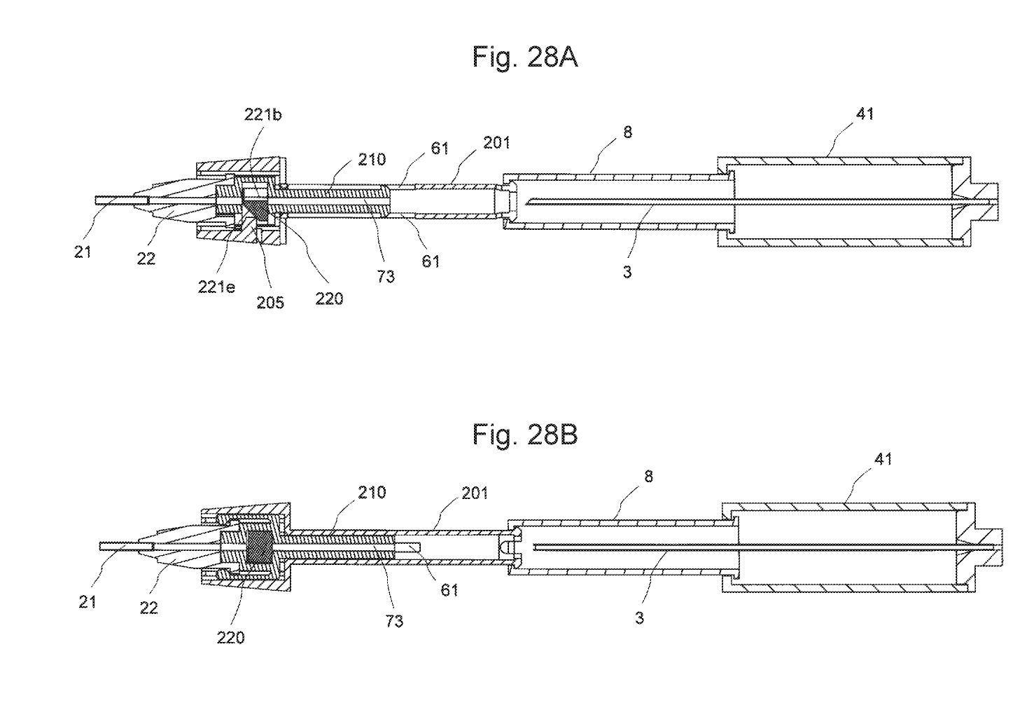

Further, as shown in FIGS. 28A and 28B, when the above-mentioned inner needle hub 41 is moved in a direction away from the above-mentioned outer needle 21 along with the axial direction, the outer pipe 201 moves relative to the inner pipe 210, the projection 205 slides on the front face 221c, and the block body 220 is pushed up (moved) by the projection 205.

According to the above, the concave portion 221b is arranged to be placed elsewhere than on the line that extends from the through hole 73 and a communication state of the through hole 73 to be blocked by the main body 221 of the block body.

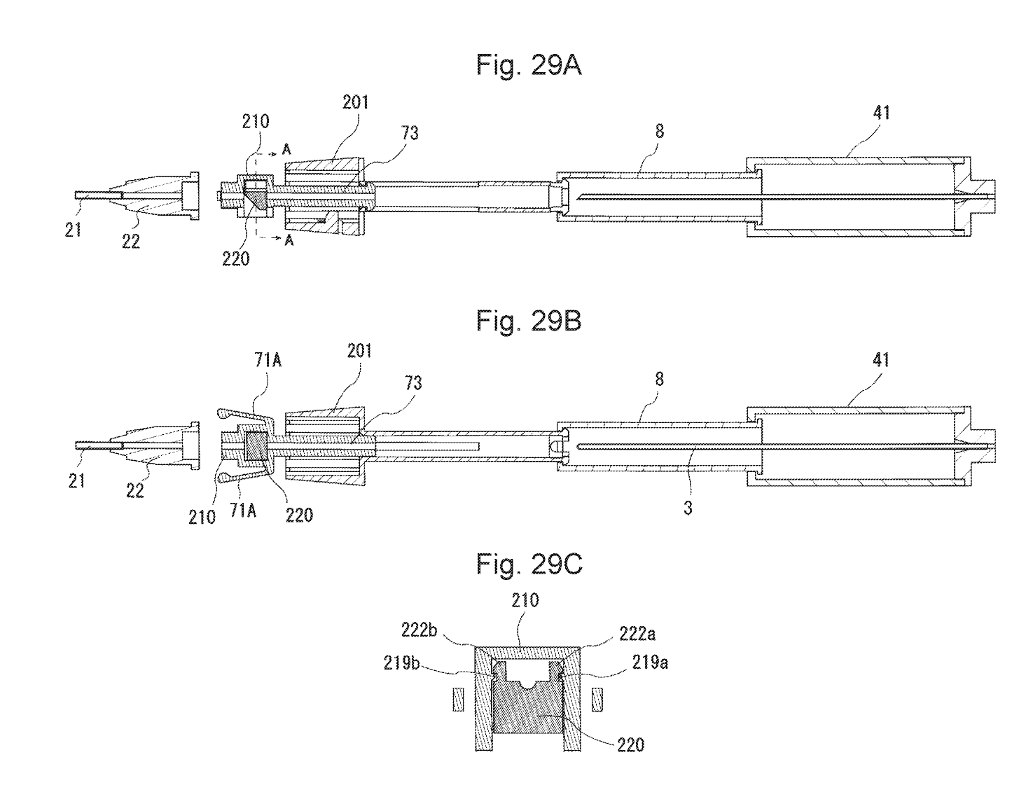

Further, when the above-mentioned inner needle hub 41 is moved in a direction away from the above-mentioned outer needle 21 along with the axial direction, as shown in FIG. 29C, the hook portions 222a, 222b of the pushed up block body 220 are engaged on the projections 219a, 219b whereby the block body is fixed and a blocked state is sustained.

Therefore, after the hook portions 222a, 222b are engaged on the projections 219a, 219b and the through hole 73 is blocked by the block body 220, even if the force of moving the inner needle hub 41 to the catheter 2 side is applied, the block body 220 inhibits the movement of the inner needle 3 and the inner needle 3 does not return into the outer needle 21 gain.

In the third embodiment, as in the first embodiment, when the above-mentioned inner pipe 210 is pulled from the above-mentioned outer pipe 201, the above-mentioned two arms 71A are opened as shown in FIG. 29B, and the retention of the outer needle hub 22 by the above-mentioned two arms 71A is released. That is to say, by separating the above-mentioned inner needle hub 41 from the above-mentioned outer needle 21 along the axial direction and expanding the above-mentioned syringe 4, the above-mentioned inner needle 3 pulled from the above-mentioned catheter 2 is accommodated inside the above-mentioned outer pipe 201 and the inner needle hub 41, then the outer needle hub 22 is released from the state where the outer needle hub 22 is retained by the above-mentioned four arms 71A and arm opening/closing parts 62.

Accordingly, whilst indwelling the above-mentioned catheter 2 (outer needle 21) in a blood vessel, the above-mentioned inner needle 3 is pulled from the above-mentioned outer needle 21 and accommodated inside the above-mentioned inner needle hub 41, and the above-mentioned outer needle hub 22 is removed from the above-mentioned syringe 4.

Thus, only by pulling the above-mentioned inner needle 3 from the above-mentioned outer needle 21, the above-mentioned puncture device 200 can accommodate the above-mentioned inner needle 3 in the above-mentioned inner needle hub 41, and the above-mentioned outer needle hub 22 can be removed from the above-mentioned inner needle hub 41.

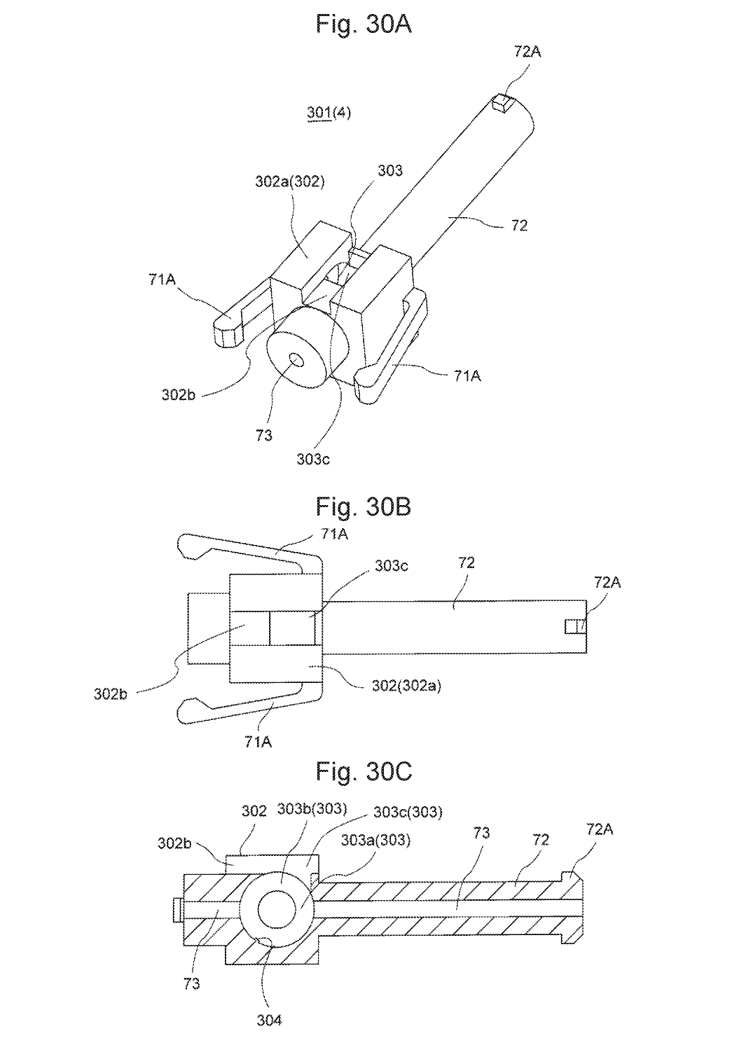

Next, a fourth preferred embodiment will be described with reference to FIGS. 30 to 36.

A through hole block means in this embodiment includes a block body in which a through hole for inserting the inner needle is formed, a space formed in the inner pipe, in which the block body is turnably accommodated. When an outer pipe moves with respect to an inner pipe, the block body turns inside the space by a projection formed on an inner surface of the outer pipe and an insertion of the inner needle is blocked.

In addition, while the block body is arranged to be movable in an upper/lower direction in the third embodiment, on the other hand the fourth embodiment differs in that the block body is arranged to be turnable. It should be noted that the same parts or corresponding parts are designated by the same reference signs as in the first or the third preferred embodiment, and will not be described further in detail.

Next, the inner pipe 301 is explained below.

A puncture device 300 of the fourth preferred embodiment differs in the inner pipe 301 and the block body 310 from the inner pipe 210 and the block body 220 of the third preferred embodiment. The outer pipe has the same construction with the third preferred embodiment.

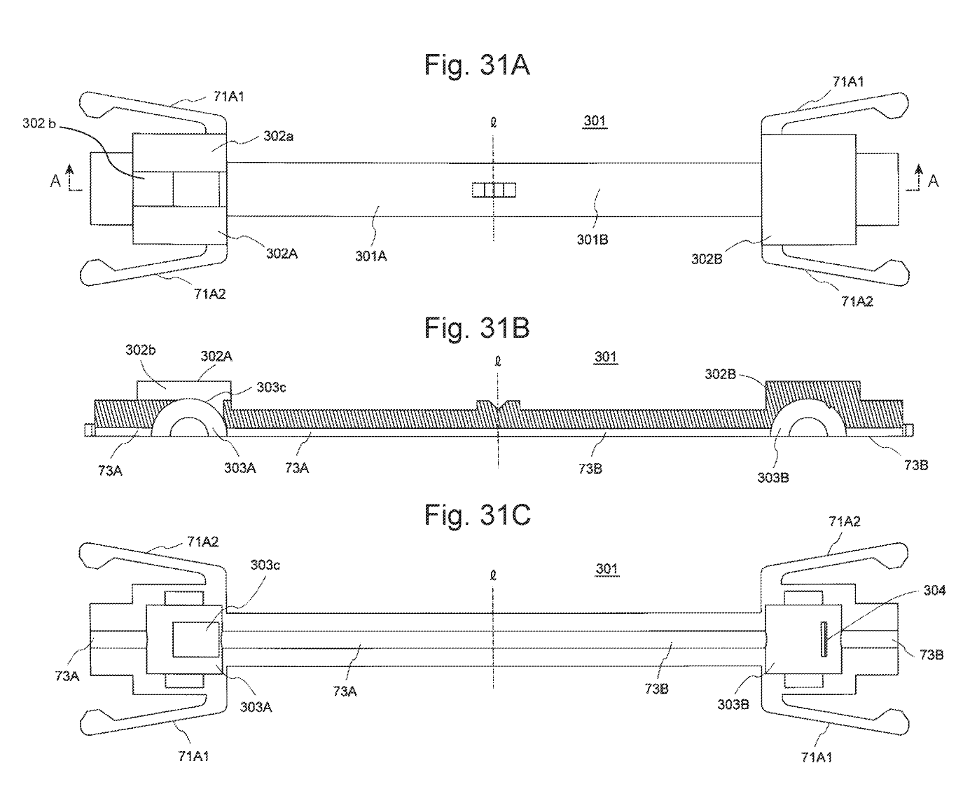

As shown in FIGS. 30A, 30B, 30C, 31A, 31B and 31C, the inner pipe 301 has a rectangular head part 302 provided with the above-mentioned two arms 71A, a shaft 72 having a diameter smaller than that of the above-mentioned head part 302 and a through hole 73 which penetrates the centers of the above-mentioned head part 302 and the above-mentioned shaft 72, and through which the above-mentioned inner needle 3 is inserted.

As shown in FIGS. 30A, 30B, 30C, 31A, 31B and 31C, a space 303 which connect to the through hole 73 in which inner needle 3 is inserted is formed inside the head part 302.

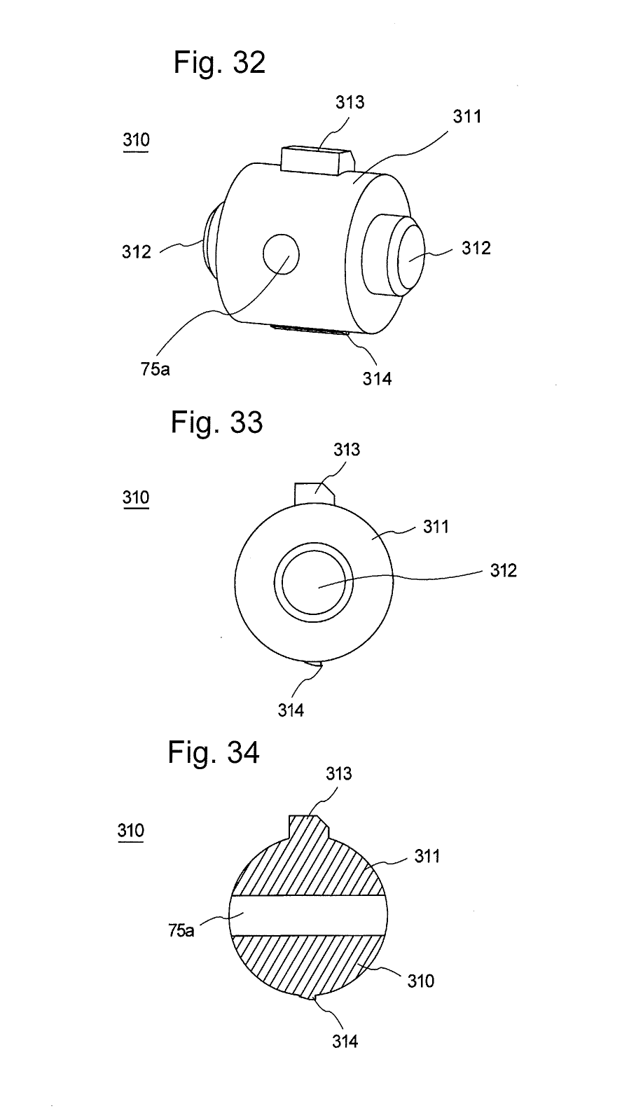

In addition, as shown in FIG. 32, the space 303 is for accommodating the block body 310 having the through hole 73 in which inner needle 3 is inserted. The space 303 is formed in cylindrical shape with a larger diameter than an outside diameter of the block body 310 so that the block body 310 can move in the space 303.

The space 303 is provided with a first cylindrical space part 303a which accommodates the block body 310, and a second cylindrical space part 303b which is formed on the right and the left of the first cylindrical space part 303a. The second cylindrical space part 303b is for a space which accommodates a rotary axis of the block body 310.

A concave portion 302b is formed on an upper wall 302a of the head part 302 in a front-rear direction (in an axial direction of the through hole 73). An opening 303c of the space 303 is formed on a bottom of the concave portion 302b.

Further, a projection 304 is formed on an inner wall surface of the head part 302, which forms the space part 303a. The projection 304 is for engaging with the block body 310 and extends toward the direction orthogonal to the axial direction of the through hole 73.

As shown in FIGS. 31A, 31B and 31C, the inner pipe 301 is a component in which an upper part 301A (left-hand side) and a lower part 301B (right-hand side) which are on opposite sides of a central line 1 are integrally formed. It is formed by folding the above-mentioned upper part 301A and the above-mentioned lower part 301B along a folding line (central line 1).

Further, grooves 73A and 73B whose cross sections are in the shape of a semicircle are formed along the axial direction of the above-mentioned inner pipe 301 in the centers of the above-mentioned upper part 301A and the above-mentioned lower part 301B, respectively. When the above-mentioned lower part 301A and the above-mentioned upper part 301B are folded, these grooves 73A and 73B form one through hole 73.

The head part 302A and the head part 302B are formed on end portions of the upper part 301A and the lower part 301B respectively. Concave portions 303A and 303B whose cross sections are in the shape of a semicircle are formed on the head part 302A and the head part 302B respectively. When the above-mentioned upper part 301A and the above-mentioned lower part 301B are folded, one space 303 is formed.

Arms 71A1, 71A2 are formed at the head part 302A and the head part 302B, respectively. When the above-mentioned upper part 301A and the above-mentioned lower part 301B are folded, two arms 71A are formed at the head part 302.

Next, the block body 310 is explained below.

As shown in FIGS. 31 to 33, the block body 310 is provided with a cylindrical main body 311, and shafts 312 formed on both end faces of the main body 311.

In addition, a through hole 75a for insertion of the inner needle 3 is formed in the block body 311 in the direction orthogonal to the axial direction of the main body 311.

A projection 313 projecting to the concave portion 302b from the opening 303c of the space 303 is formed on a peripheral surface of the cylindrical main body 311.

Since a tip portion of the projection 313 projects in the concave portion 302b, when an inner pipe 301 moves with respect to an outer pipe, the projection 205 (See. FIGS. 22B and 22C) contact to the projection 313, which rotates the block body 310.

A hook portion 314 is formed on the peripheral surface of the cylindrical main body 311, which is engaged with the projection 304. When the block body 310 rotates in the space 313, the hook portion 314 is engaged with the projection 304 whereby the block body 310 is fixed in the space 313 and a rotation of the block body 310 is inhibited.

As described above, a projection 205 of the outer pipe 201 rotates the block body 310 from a position where the inner needle 3 is inserted to a position where the inner needle 3 is not inserted and the communication state of the through hole 73 is blocked.

Next, work and operation of the puncture device 300 in accordance with the above structured fourth preferred embodiment will be described.

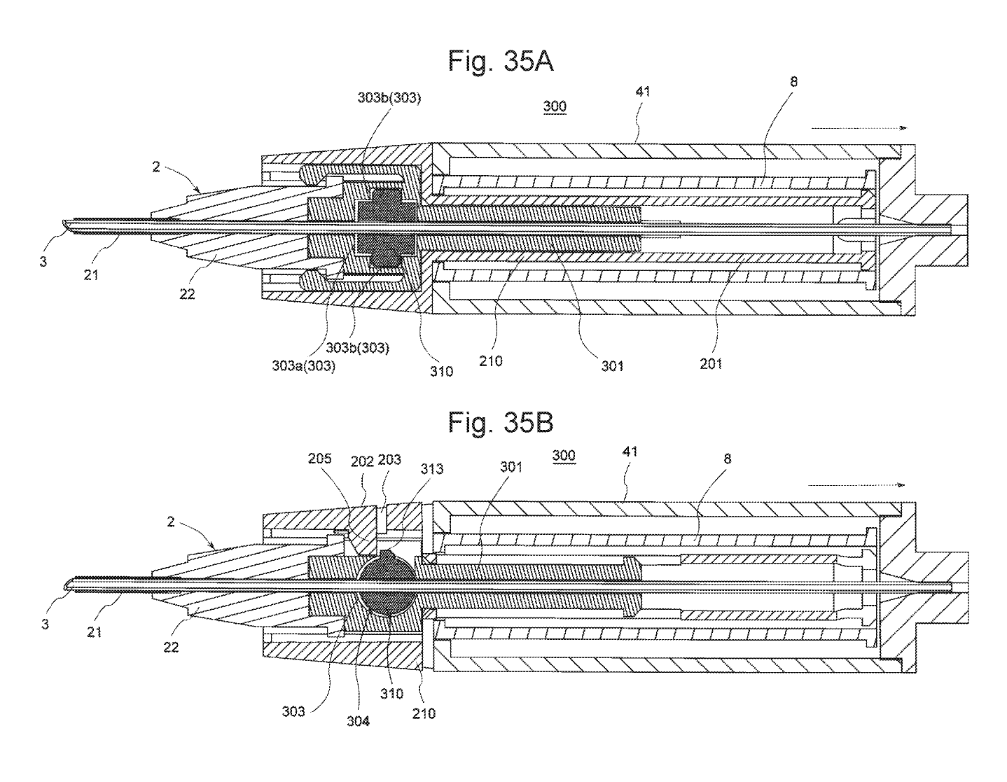

Firstly, the protector 5 is removed from the puncture device 300, and the catheter 2 and the inner needle 3 are exposed as shown in FIGS. 35A and 35B. Further, the above-mentioned outer needle 21 and the above-mentioned inner needle 3 are punctured to a blood vessel (patient's body).

Then, in order to indwell the above-mentioned outer needle 21, the above-mentioned inner needle hub 41 is moved along the axial direction shown by the arrows in FIGS. 35A and 35B and in a direction away from the above-mentioned outer needle 21 (pull-out operation is performed). As the inner needle hub 41 for retaining the rear end portion (base portion) of the inner needle 3 is moved along the axial direction, the inner needle 3 similarly moves along the axial direction. Further, the inner needle 3 is pulled from the outer needle 21, and the thus pulled inner needle 3 is covered with the inner pipe 301, the outer pipe 210, and the inner needle hub 41.

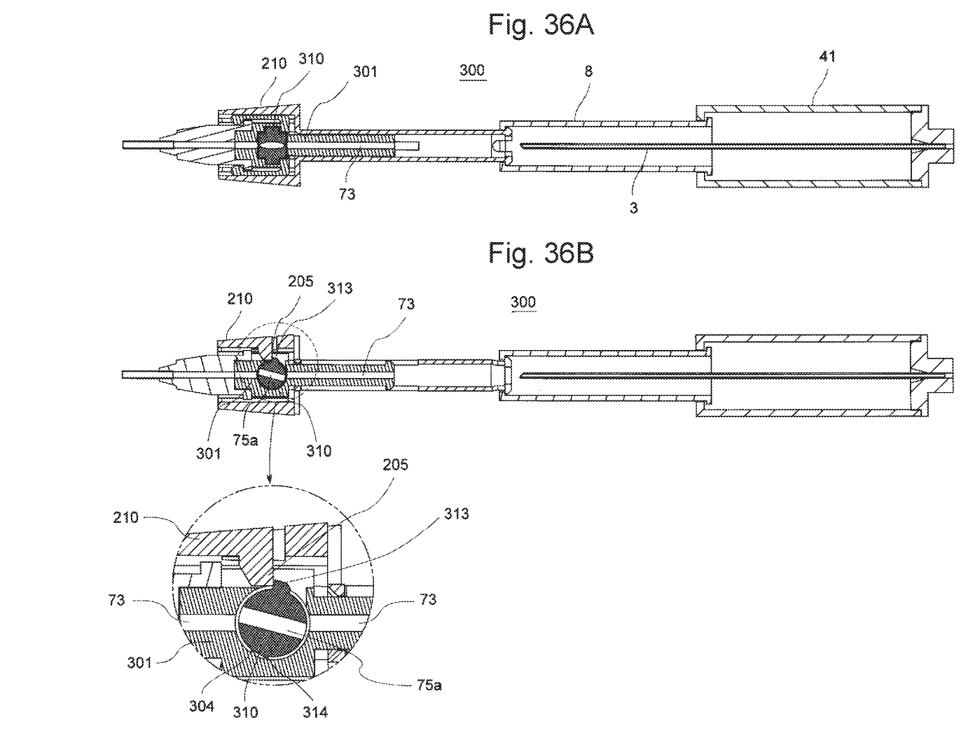

Further, when the above-mentioned inner needle hub 41 is moved in a direction away from the above-mentioned outer needle 21 along with the axial direction, the inner pipe 301 moves inside the outer pipe 210 (the outer pipe 201 moves relative to the inner pipe 301), the projection 205 abuts on the projection 313 of the block body 310 and the block body 310 is rotated (see. FIGS. 36A and 36B).

In accordance with the rotation of the block body 310, the through hole 75a of the block body 310 is also rotated, and the through hole 75a is placed elsewhere than on the line that extends from the through hole 73 which causes a communication state of the through hole 73 to be blocked (see. FIG. 36B).

Therefore, even if the force of moving the inner needle hub 41 to the catheter 2 side is applied, the block body 310 inhibits the movement of the inner needle 3 and the inner needle 3 does not return into the outer needle 21 gain.

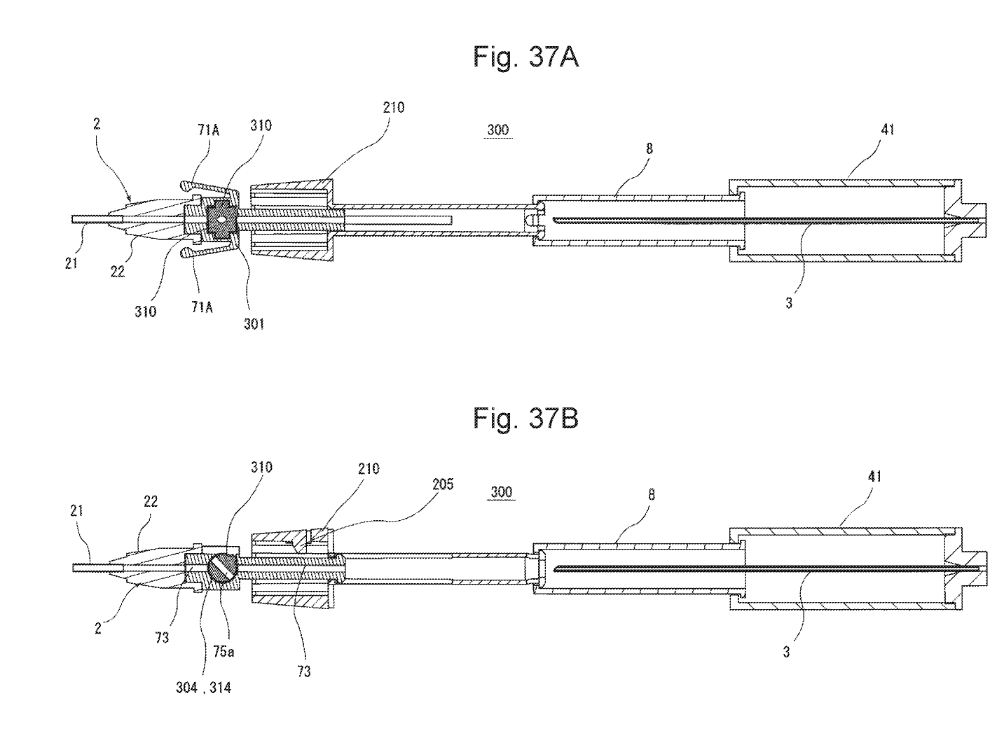

Further, when the inner needle hub 41 is moved along the axial direction away from the outer needle 21, as shown in FIGS. 37A and 37B, the hook portion 314 of the block body 310 is engaged with the projection 304 whereby the block body 310 is fixed, a blocked state is sustained, and a rotation of the block body 310 is inhibited.

After the hook portion 314 of the block body 310 is engaged with the projection 304, since the block body 310 is not rotated and the through hole 73 is surely blocked, even if the force of moving the inner needle hub 41 to the catheter 2 side is applied, the block body 310 inhibits the movement of the inner needle 3 and the inner needle 3 does not return into the outer needle 21 gain.

In the fourth embodiment as shown in FIGS. 37A and 37B, when the above-mentioned inner pipe 301 is pulled from the above-mentioned outer pipe 201, the above-mentioned two arms 71A are opened, and the retention of the outer needle hub 22 by the above-mentioned two arms 71A is released.

Accordingly, whilst the above-mentioned catheter 2 (outer needle 21) indwelling in a blood vessel, the above-mentioned inner needle 3 is pulled from the above-mentioned outer needle 21 and accommodated inside the above-mentioned inner needle hub 41, and the above-mentioned outer needle hub 22 is removed from the above-mentioned syringe 4.

Thus, only by pulling the above-mentioned inner needle 3 from the above-mentioned outer needle 21, the above-mentioned puncture device 1 can accommodate the above-mentioned inner needle 3 in the above-mentioned inner needle hub 41, and the above-mentioned outer needle hub 22 can be removed from the above-mentioned inner needle hub 41.

### DESCRIPTION OF THE REFERENCE NUMERALS

1, 100 puncture device
2, 101 catheter
21, 102 outer needle
22, 102a outer needle hub
3, 103 inner needle
4, 104 syringe
5 protector
6, 107 outer pipe (cylinder body)
7, 108 inner pipe (cylinder body)
7A upper part
7B lower part
41, 105 inner needle hub
42, 106 plug
42a, 106a needle retaining part
61, 107b groove
62, 107c arm opening/closing part (gripping means)
107d groove
107d1 linear portion
107d2 spiral portion
71, 108B head part
71A, 108A arm (gripping means)
72, 108C shaft
72A projection
72B, 108E standing piece
73, 108D through hole
108F projecting part
200 puncture device
201 outer pipe
205 projection
210 inner pipe
212 concave space
219a, 219b projection
220 block body
222a, 222b hook portion
300 puncture device
301 inner pipe
303 space
304 projection (engaging part)
310 block body
313 projection
314 hook portion (part to be engaged)

1. A puncture device comprising an outer needle, an outer needle hub for retaining a base portion of said outer needle, an inner needle whose tip portion is inserted in said outer needle, a cylindrical syringe for retaining a base portion of said inner needle, wherein the syringe is provided with an inner needle hub for retaining a base portion of said inner needle, a gripping means for gripping said outer needle hub, and a cylinder body fitted inside the inner needle hub so as to be freely moveable, wherein the cylinder body is provided with an outer pipe fitted inside the inner needle hub so as to be freely moveable, and inner pipe fitted inside said outer pipe so as to be freely moveable and having a through hole for inserting the inner needle,
wherein either the inner pipe or the inner pipe and the outer pipe is provided with through hole block means for blocking a communicated state of the through hole, which makes at least part of the through hole move from a position where the inner needle is inserted to a position where the inner needle is blocked, and
wherein insertion of the inner needle is blocked by moving the through hole of the inner pipe with the through hole block means.

2. A puncture device as claimed in claim 1, wherein the through hole block means comprises a block body in which a through hole for inserting the inner needle is formed and a space portion formed in the inner pipe, which movably accommodates the block body, and
when a tip of the inner needle is pulled out through the block body, the block body moves in the space part by the block body's own weight and insertion of the inner needle is blocked.

3. A puncture device as claimed in claim 1, wherein the inner pipe further comprises a standing piece which rises with being forced out by the inner needle in the through hole and engages with the outer pipe, and
when the inner needle is pulled out passing by the standing piece, the standing piece is disengaged with the outer pipe, and the disengaged standing piece blocks insertion of the inner needle.

4. A puncture device as claimed in claim 2, wherein the inner pipe further comprises a standing piece which rises with being forced out by the inner needle in the through hole and engages with the outer pipe, and
when the inner needle is pulled out passing by the standing piece, the standing piece is disengaged with the outer pipe, and the disengaged standing piece blocks insertion of the inner needle.

5. A puncture device as claimed in claim 2, wherein at least the inner pipe and the block body are formed of a synthetic resin material.

6. A puncture device as claimed in claim 1, wherein the through hole block means comprises a groove having at least a spiral portion, which is formed on an inner surface of the outer pipe or an outer surface of the inner pipe and a projecting part being movable in the groove, which is formed on an outer surface of the inner pipe or an inner surface of the outer pipe, the through hole on the inner pipe is formed at away from the central axis of the inner pipe, and
when the outer pipe moves relative to the inner pipe, the inner pipe and the outer pipe relatively rotate by engagement of the groove and the projecting part so that the through hole of the inner pipe moves from a position where the inner needle is inserted to a position where the inner needle is blocked in such a manner that insertion of the inner needle is blocked.

7. A puncture device as claimed in claim 6, wherein the inner pipe further comprises a standing piece which rises with being forced out by the inner needle in the through hole and engages with the outer pipe, and
when the inner needle pulled out passing by the standing piece, the standing piece is disengaged with the outer pipe, and the disengaged standing piece blocks insertion of the inner needle.

8. A puncture device as claimed in claim 6, wherein at least the outer pipe and the inner pipe are formed of a synthetic resin material.

9. A puncture device as claimed in claim 1, wherein the through hole block means comprises a projection formed on an inner surface of the outer pipe, a block body in which a through hole for inserting the inner needle is formed and a space portion formed in the inner pipe, which movably accommodates the block body, and when the outer pipe moves relative to the inner pipe, the block body is moved in the space portion by the projection formed on the inner surface of the outer pipe in such a manner that insertion of the inner needle is blocked.

10. A puncture device as claimed in claim 9, wherein a part to be engaged formed on the block body is engaged to an engaging part formed in the inner pipe so that movement of the block body is prevented and a state that the insertion of the inner needle is blocked is kept.

11. A puncture device as claimed in claim 9, wherein the outer pipe, the inner pipe and the block body are formed of a synthetic resin material.
